(19) United States
(12) Patent Application Publication
(10) Pub. No.: US 2022/0072307 A1
(43) Pub. Date: Mar. 10, 2022

(54) TECHNIQUES FOR STIMULATION ARTEFACT ELIMINATION

(71) Applicant: Cochlear Limited, Macquarie University, NSW (AU)

(72) Inventors: Ryan Orin MELMAN, Macquarie University (AU); Todd LUPTON, Macquarie University (AU); Paul Michael CARTER, Macquarie University (AU); Shaun KUMAR, Macquarie University (AU)

(21) Appl. No.: 17/416,683

(22) PCT Filed: May 5, 2020
(86) PCT No.: PCT/IB2020/054266

§ 371 Date: Jun. 21, 2021

Related U.S. Application Data
(60) Provisional application No. 62/844,079, filed on May 6, 2019.

(51) Int. Cl. A61N 1/36; A61B 5/388

(52) U.S. Cl. CPC A61N 1/36039; A61B 5/388

(57) ABSTRACT

A method of evaluating a neural response, comprising applying electrical stimulation to a recipient, obtaining from read electrodes read data resulting from the applied stimulation, obtaining an artefact model based at least in part on the read data, and obtaining neural response data by comparing the read data to the artefact model.

### CROSS-REFERENCE TO RELATED APPLICATIONS

This application claims priority to U.S. Provisional Application No. 62/844,079, entitled TECHNIQUES FOR STIMULATION ARTEFACT ELIMINATION, filed on May 6, 2019, naming Ryan Orin MELMAN of Macquarie University, Australia as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. A hearing prosthesis can be a cochlear implant.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from hearing loss typically receive an acoustic hearing aid. Conventional hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve. Cases of conductive hearing loss typically are treated by means of bone conduction hearing aids. In contrast to conventional hearing aids, these devices use a mechanical actuator that is coupled to the skull bone to apply the amplified sound.

In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound.

Many devices, such as medical devices that interface with a recipient, have structural and/or functional features where there is utilitarian value in adjusting such features for an individual recipient. The process by which a device that interfaces with or otherwise is used by the recipient is tailored or customized or otherwise adjusted for the specific needs or specific wants or specific characteristics of the recipient is commonly referred to as fitting.

### SUMMARY

In accordance with an exemplary embodiment, there is a method, comprising applying electrical stimulation to a recipient, obtaining from read electrodes read data resulting from the applied stimulation, obtaining an artefact model based at least in part on the read data and obtaining neural response data by comparing the read data to the artefact model.

In accordance with another exemplary embodiment, there is a method that includes developing a recipient-specific electrical stimulation artefact model.

In accordance with an another exemplary embodiment, there is an electrical response stimulation measurement system, comprising an input sub-system configured to receive first data based on a signal response to stimulation applied to a person; and a processor and/or chip assembly configured to develop a model based at least in part on the received first data and to extrapolate a biological signal based on a comparison of the model and the received first data.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIG. 1B depicts a side view of the cochlear implant 100 outside of the recipient;

FIGS. 2A and 2B are side views of an embodiment of an insertion guide for implanting a cochlear implant electrode assembly such as the electrode assembly illustrated in FIG. 1;

FIGS. 3A and 3B are side and perspective views of an electrode assembly extended out of an embodiment of an insertion sheath of the insertion guide illustrated in FIG. 2;

FIGS. 4A-4E are simplified side views depicting the position and orientation of a cochlear implant electrode assembly insertion guide tube relative to the cochlea at each of a series of successive moments during an exemplary implantation of the electrode assembly into the cochlea;

FIGS. 5-9 are exemplary system components of an exemplary embodiment;

FIG. 10 provides a conceptual electrical schematic associated with electrodes inside a cochlea;

FIGS. 11 and 12 provide some exemplary data in the form of charts;

FIG. 13 provides an exemplary flowchart for an exemplary method;

FIGS. 14, 16, 17 and 18 provide some conceptual charts that are utilized to describe some of the embodiments herein;

FIGS. 15 and 15A provide an exemplary flowchart for an exemplary method;

FIG. 19 provides an exemplary flowchart for an exemplary method;

FIG. 20 provides another conceptual electrical schematic associated with electrodes inside a cochlea;

FIGS. 21, 22, and 23 provide some additional conceptual charts that are utilized to describe some of the embodiments herein;

FIGS. 24, 25, 26, and 27 provide some exemplary flowcharts for an exemplary method; and

FIGS. 28 and 29 provide some schematics of exemplary systems according to some exemplary embodiments.

### DETAILED DESCRIPTION

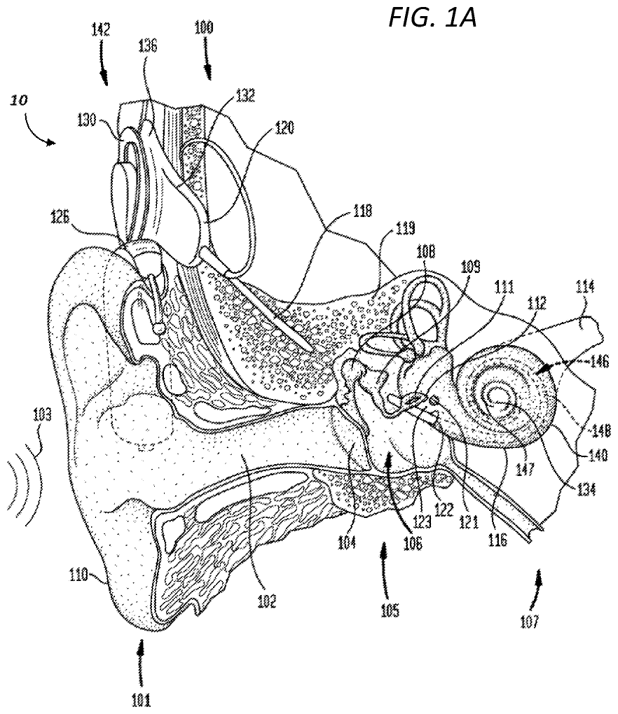

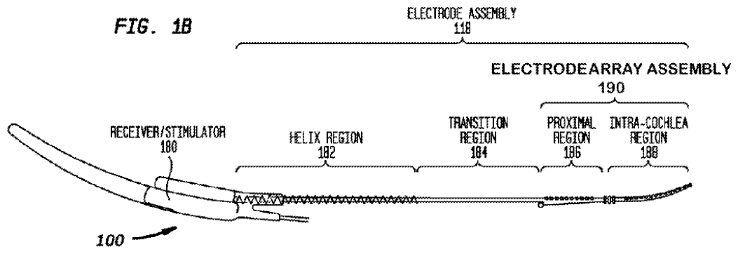

FIG. 1A is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. Additionally, it is noted that the teachings detailed herein are also applicable to other types of hearing prostheses, such as by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing. Accordingly, any disclosure herein with regard to one of these types of hearing prostheses corresponds to a disclosure of another of these types of hearing prostheses or any medical device for that matter, unless otherwise specified, or unless the disclosure thereof is incompatible with a given device based on the current state of technology. Thus, the teachings detailed herein are applicable, in at least some embodiments, to partially implantable and/or totally implantable medical devices that provide a wide range of therapeutic benefits to recipients, patients, or other users, including hearing implants (with or without an implanted microphone, vestibular stimulators, vagal stimulators, auditory brain stimulators, pacemakers, visual prostheses (e.g., bionic eyes), sensors, drug delivery systems, defibrillators, functional electrical stimulation devices including closed loop spinal stimulators, etc.

In view of the above, it is to be understood that at least some embodiments detailed herein and/or variations thereof are directed towards a body-worn sensory supplement medical device (e.g., the hearing prosthesis of FIG. 1A, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is rechargeable via the transcutaneous link.

In the illustrative arrangement of FIG. 1A, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments, electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

FIG. 1B is a side view of a cochlear implant 100 without the other components of system 10 (e.g., the external components). Cochlear implant 100 comprises a receiver/stimulator 180 and an electrode assembly or lead 118. Electrode assembly 118 includes a helix region 182, a transition region 184, a proximal region 186, and an intra-cochlear region 188. Proximal region 186 and intra-cochlear region 188 form an electrode array assembly 190. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the electrode array assembly 190. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected through lead 118 to receiver/stimulator 180, through which respective stimulating electrical signals for each electrode contact 148 travel.

Electrode array 146 may be inserted into cochlea 140 with the use of an insertion guide. It is noted that while the embodiments detailed herein are described in terms of utilizing an insertion guide or other type of tool to guide the array into the cochlea, in some alternate insertion embodiments, a tool is not utilized. Instead, the surgeon utilizes his or her fingertips or the like to insert the electrode array into the cochlea. That said, in some embodiments, alternate types of tools can be utilized other than and/or in addition to insertion guides. By way of example only and not by way of limitation, surgical tweezers like can be utilized. Any device, system, and/or method of inserting the electrode array into the cochlea can be utilized according to at least some exemplary embodiments.

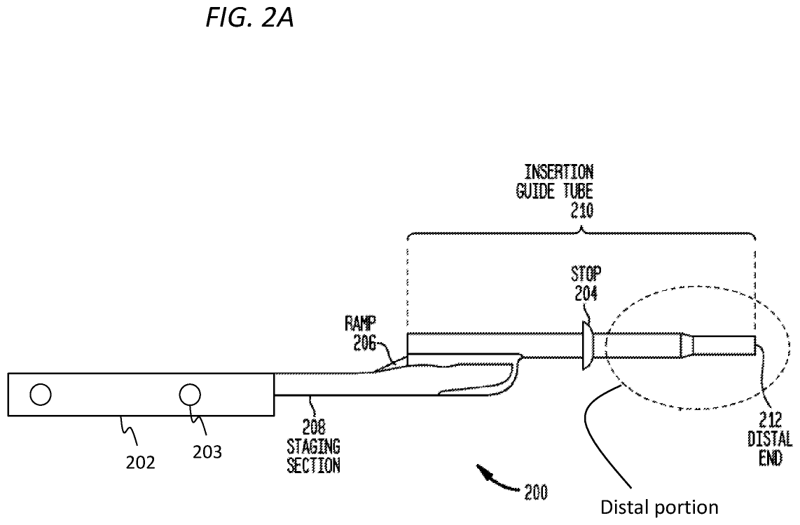

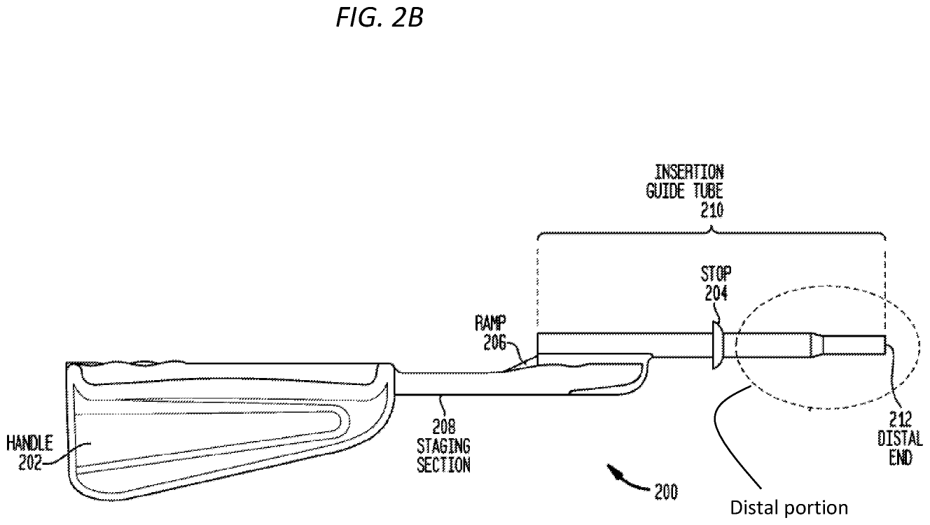

FIG. 2A presents a side view of an embodiment of an insertion guide for implanting an elongate electrode assembly generally represented by electrode assembly 145 (corresponding to assembly 190 of FIG. 1B) into a mammalian cochlea, represented by cochlea 140. The illustrative insertion guide, referred to herein as insertion guide 200, includes an elongate insertion guide tube 210 configured to be inserted into cochlea 140 and having a distal end 212 from which an electrode assembly is deployed. Insertion guide tube 210 has a radially-extending stop 204 that may be utilized to determine or otherwise control the depth to which insertion guide tube 210 is inserted into cochlea 140. (It is briefly noted that while some of the description herein relates to methods and systems related to the temporal period associated with or shortly after insertion (some embodiments are such that the methods herein are executed in the surgery room just after implantation), other embodiments include implementing the teachings and/or systems herein post-surgery/post-implantation, including days or weeks or months or years or decades after implantation. Accordingly, unless otherwise noted, any disclosure herein of a method and/or system applies to a fully implanted prosthesis that has been activated and/or is about to be activated for stimulation purposes to provide stimulation for the intended purpose of the implanted device.)

Insertion guide tube 210 is mounted on a distal region of an elongate staging section 208 on which the electrode assembly is positioned prior to implantation. A robotic arm adapter 202 is mounted to a proximal end of staging section 208 to facilitate attachment of the guide to a robot, which adapter includes through holes 203 through which bolts can be passed so as to bolt the guide 200 to a robotic arm, as will be detailed below. During use, electrode assembly 145 is advanced from staging section 208 to insertion guide tube 210 via ramp 206. After insertion guide tube 210 is inserted to the appropriate depth in cochlea 140, electrode assembly 145 is advanced through the guide tube to exit distal end 212 as described further below.

FIG. 2B depicts an alternate embodiment of the insertion guide 200, that includes a handle 202 that is ergonomically designed to be held by a surgeon. This in lieu of the robotic arm adapter 202.

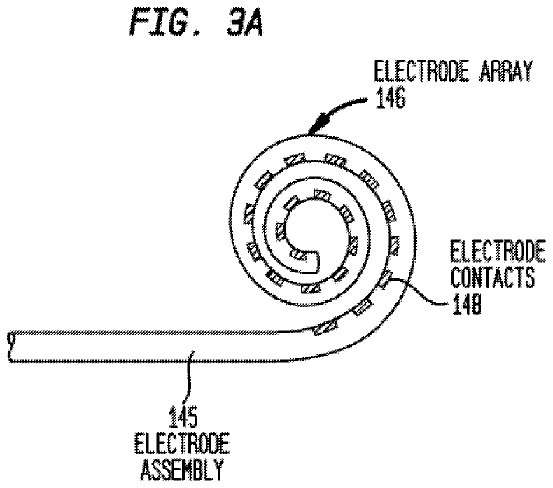

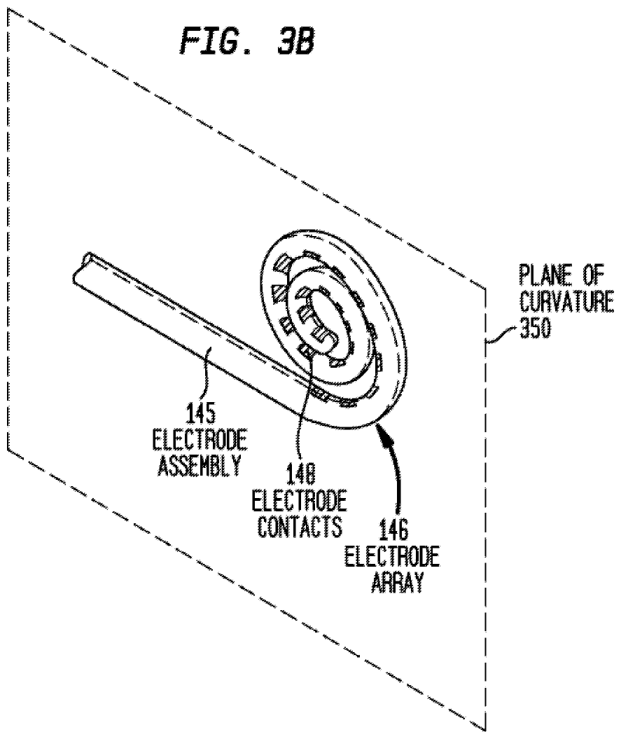

FIGS. 3A and 3B are side and perspective views, respectively, of representative electrode assembly 145. As noted, electrode assembly 145 comprises an electrode array 146 of electrode contacts 148. Electrode assembly 145 is configured to place electrode contacts 148 in close proximity to the ganglion cells in the modiolus. Such an electrode assembly, commonly referred to as a perimodiolar electrode assembly, is manufactured in a curved configuration as depicted in FIGS. 3A and 3B. When free of the restraint of a stylet or insertion guide tube, electrode assembly 145 takes on a curved configuration due to it being manufactured with a bias to curve, so that it is able to conform to the curved interior of cochlea 140. As shown in FIG. 3B, when not in cochlea 140, electrode assembly 145 generally resides in a plane 350 as it returns to its curved configuration. That said, it is noted that embodiments of the insertion guides detailed herein and/or variations thereof can be applicable to a so-called straight electrode array, which electrode array does not curl after being free of a stylet or insertion guide tube, etc., but instead remains straight.

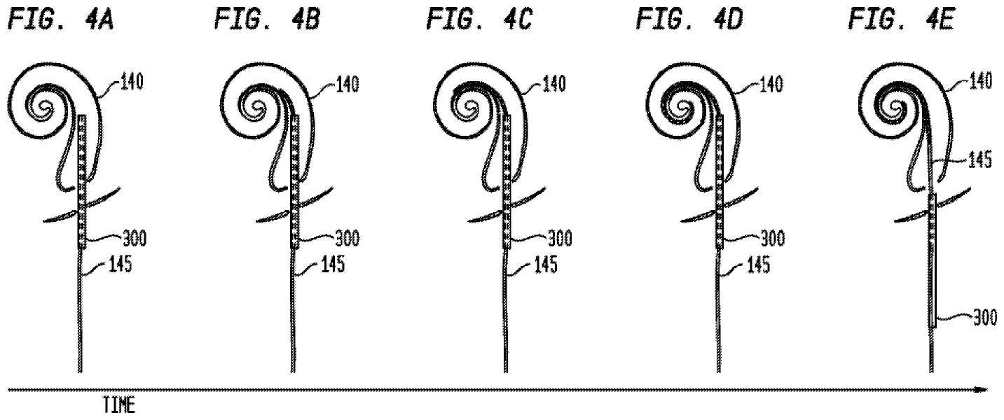

FIGS. 4A-4E are a series of side-views showing consecutive exemplary events that occur in an exemplary implantation of electrode assembly 145 into cochlea 140. Initially, electrode assembly 145 and insertion guide tube 310 are assembled. For example, electrode assembly 145 is inserted (slidingly or otherwise) into a lumen of insertion guide tube 300. The combined arrangement is then inserted to a predetermined depth into cochlea 140, as illustrated in FIG. 4A. Typically, such an introduction to cochlea 140 is achieved via cochleostomy 122 (FIG. 1) or through round window 121 or oval window 112. In the exemplary implantation shown in FIG. 4A, the combined arrangement of electrode assembly 145 and insertion guide tube 300 is inserted to approximately the first turn of cochlea 140.

As shown in FIG. 4A, the combined arrangement of insertion guide tube 300 and electrode assembly 145 is substantially straight. This is due in part to the rigidity of insertion guide tube 300 relative to the bias force applied to the interior wall of the guide tube by pre-curved electrode assembly 145. This prevents insertion guide tube 300 from bending or curving in response to forces applied by electrode assembly 145, thus enabling the electrode assembly to be held straight, as will be detailed below.

As noted, electrode assembly 145 is biased to curl and will do so in the absence of forces applied thereto to maintain the straightness. That is, electrode assembly 145 has a memory that causes it to adopt a curved configuration in the absence of external forces. As a result, when electrode assembly 145 is retained in a straight orientation in guide tube 300, the guide tube prevents the electrode assembly from returning to its pre-curved configuration. This induces stress in electrode assembly 145. Pre-curved electrode assembly 145 will tend to twist in insertion guide tube 300 to reduce the induced stress. In the embodiment configured to be implanted in scala tympani of the cochlea, electrode assembly 145 is pre-curved to have a radius of curvature that approximates the curvature of medial side of the scala tympani of the cochlea. Such embodiments of the electrode assembly are referred to as a perimodiolar electrode assembly, and this position within cochlea 140 is commonly referred to as the perimodiolar position. In some embodiments, placing electrode contacts in the perimodiolar position provides utility with respect to the specificity of electrical stimulation, and can reduce the requisite current levels thereby reducing power consumption.

As shown in FIGS. 4B-4D, electrode assembly 145 may be continually advanced through insertion guide tube 300 while the insertion sheath is maintained in a substantially stationary position. This causes the distal end of electrode assembly 145 to extend from the distal end of insertion guide tube 300. As it does so, the illustrative embodiment of electrode assembly 145 bends or curves to attain a perimodiolar position, as shown in FIGS. 4B-4D, owing to its bias (memory) to curve. Once electrode assembly 145 is located at the desired depth in the scala tympani, insertion guide tube 300 is removed from cochlea 140 while electrode assembly 145 is maintained in a stationary position. This is illustrated in FIG. 4E.

Conventional insertion guide tubes typically have a lumen dimensioned to allow the entire tapered electrode assembly to travel through the guide tube. Because the guide tube is able to receive the relatively larger proximal region of the electrode assembly, there will be a gap between the relatively smaller distal region of the electrode assembly and the guide tube lumen wall. Such a gap allows the distal region of the electrode assembly to curve slightly until the assembly can no longer curve due to the lumen wall.

Returning to FIGS. 3A-3B, perimodiolar electrode assembly 145 is pre-curved in a direction that results in electrode contacts 148 being located on the interior of the curved assembly, as this causes the electrode contacts to face the modiolus when the electrode assembly is implanted in or adjacent to cochlea 140. Insertion guide tube 300 retains electrode assembly 145 in a substantially straight configuration, thereby preventing the assembly from taking on the configuration shown in FIG. 3B.

It is noted that while the embodiments above disclose the utilization of an insertion tool, in some other embodiments, insertion of the electrode array is not executed utilizing an insertion tool. Moreover, in some embodiments, when an insertion tool is utilized, the insertion tool is not as intrusive as that detailed in the figures. In an exemplary embodiment, there is no distal portion of the tool. That is, the insertion tool stops at the location where the distal portion begins. In an exemplary embodiment, the tool stops at stop 204. In this regard, there is little to no intrusion of the tool into the cochlea. Any device, system, and/or method that can enable the insertion of the electrode array can be utilized in at least some exemplary embodiments.

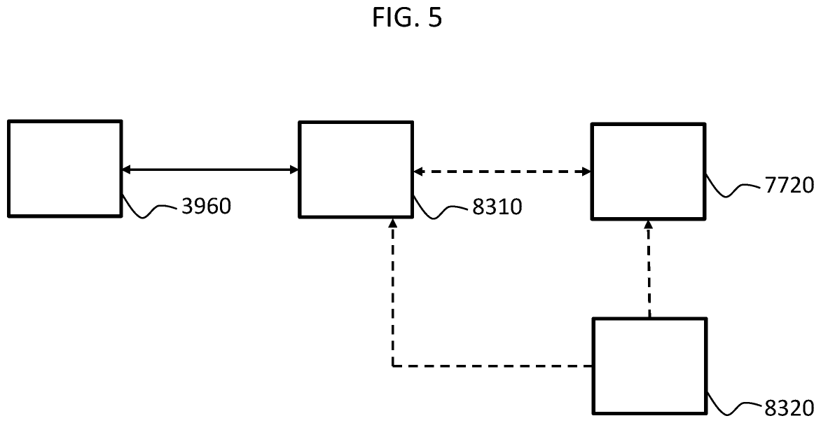

As can be recognized from the above, the electrode array can be utilized to obtain the data utilized in the methods herein, such as by way of example only and not by way of limitation, the voltages at the read electrodes, and can also be used to provide the stimulating electrode. FIG. 5 depicts an exemplary system for utilizing the cochlear implant to obtain such information. Presented in functional terms, there is a test unit 3960 in signal communication with unit 8310, which in turn is in signal communication, optionally with a unit 7720 and a unit 8320, the details of which will be described below.

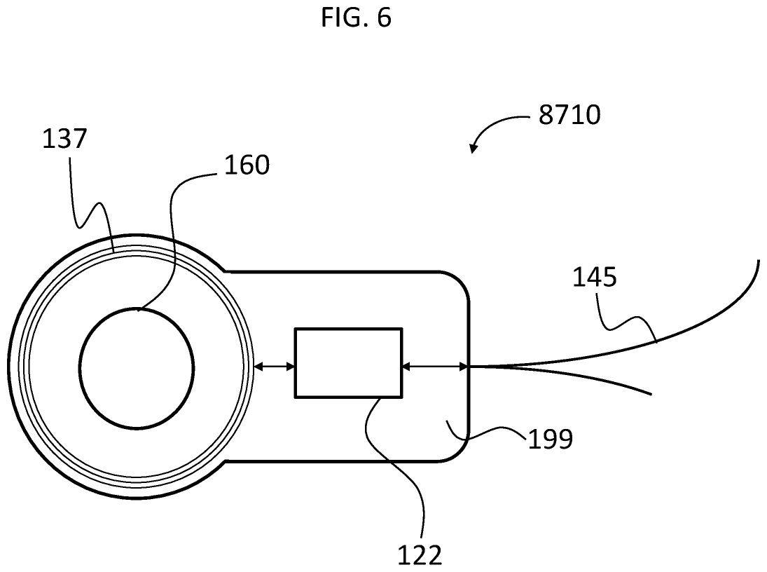

Unit 3960 can correspond to an implantable component of an electrode array, as seen in FIG. 1. More specifically, FIG. 6 depicts an exemplary high-level diagram of a receiver/stimulator 8710 (the implantable portion of 100) of a cochlear implant, looking downward. As can be seen, the receiver/stimulator 8710 includes a magnet 160 that is surrounded by a coil 137 that is in two-way communication (although in other embodiments, the communication is one-way) with a stimulator unit 122, which in turn is in communication with the electrode array 145. Receiver/stimulator 8710 further includes a cochlear stimulator unit 122, in signal communication with the coil 137. The coil 137 and the stimulator unit 122 are encased in silicon as represented by element 199. In an exemplary embodiment, receiver/stimulator 8710 is utilized as test unit 3960, and is used to implement one or more of the teachings detailed herein.

It is briefly noted that in some embodiments, the arrangement of FIG. 5 can utilize the external components of the hearing prostheses as the interface with the implant, as will be discussed in limited detail below.

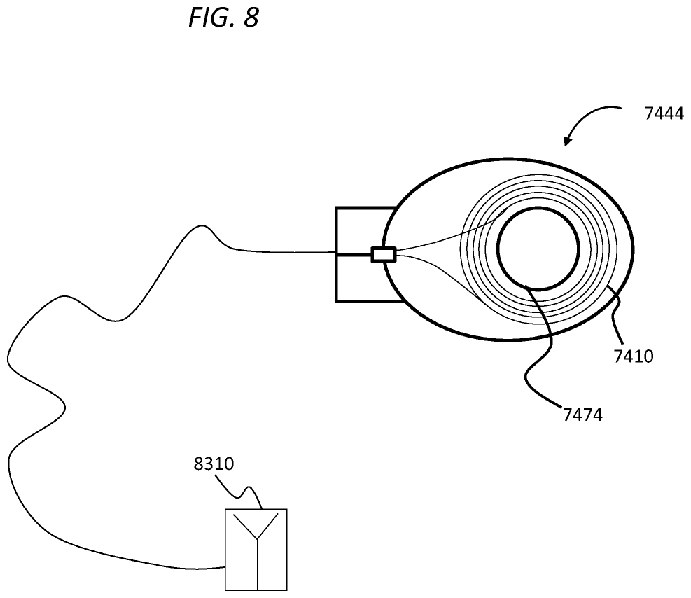

FIG. 8 depicts an exemplary RS (receiver/stimulator) interface 7444 which is presented by way of concept. An inductance coil 7410 is configured to establish a magnetic inductance field so as to communicate with the corresponding coil of the receiver-stimulator of the cochlear implant. Interface 7444 includes a magnet 7474 so as to hold the inductance coil 7410 against the coil of the receiver/stimulator of the cochlear implant in a manner analogous to how the external component of the cochlear implant is held against the implanted component, and how the coils of those respective components are aligned with one another. As can be seen, an electrical lead extends from the coil 7410 to control unit 8310, representing signal communication between interface 7444, and control unit 8310. It is noted that in an alternative embodiment, 7444 can be the external component of FIG. 1, and can have some and/or all of the functionalities just described, such that data can be obtained from the implanted portion outside of a clinical setting, such as during everyday life of the recipient.

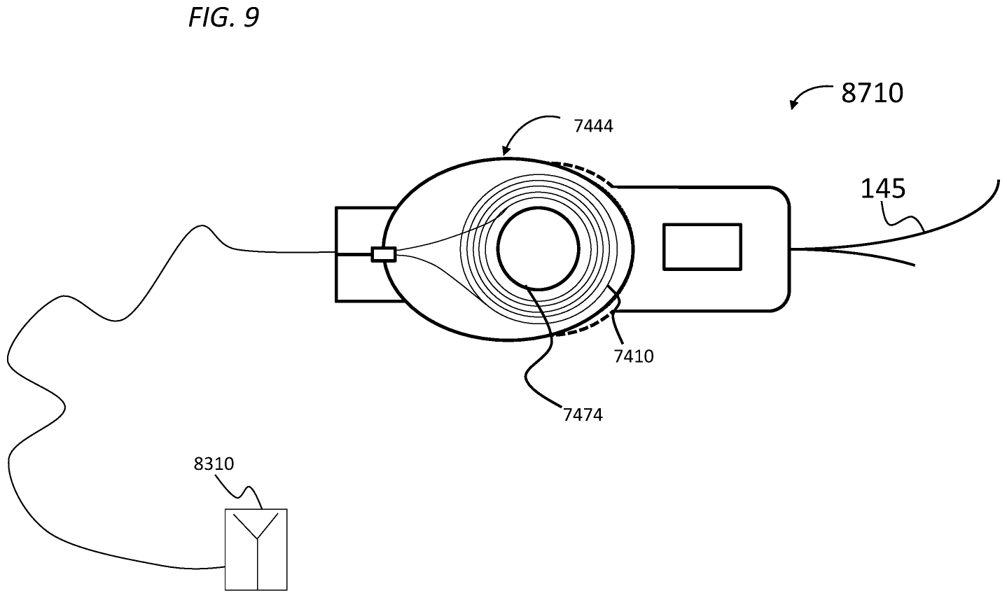

FIG. 9 depicts an exemplary embodiment of the receiver/stimulator 8710 in signal communication with the control unit 8310 via electrical lead that extends from the interface device 7444 having coil 7410 about a magnet 7474 as can be seen. The interface device 7444 communicates via an inductance field with the inductance coil of the receiver/stimulator 8710 so that the data acquired by the implantable component 8710 (receiver/stimulator) can be transferred to the control unit 8310.

Note also that in at least some alternate exemplary embodiments, control unit 8310 can communicate with the so-called “hard ball” reference electrode of the implantable component of the cochlear implant so as to enable communication of data from the receiver/stimulator 8710 to control unit 8310 and/or vice versa.

It is noted that in the embodiment of FIG. 9, control unit 8310 is in signal communication with the various other components as detailed herein, which components are not depicted in FIG. 9 for purposes of clarity.

Also functionally depicted in FIG. 5 is the optional embodiment where an electrode array insertion robotic system/actuator system 7720 and an input device 8320 is included in the system. In an exemplary embodiment, the input device 8320 could be a trigger of a handheld device that controls the actuator system 7720 and can stop and/or start the actuator for insertion of the electrode array. In an exemplary embodiment, the input device 8320 could be a trigger on the tool 8200.

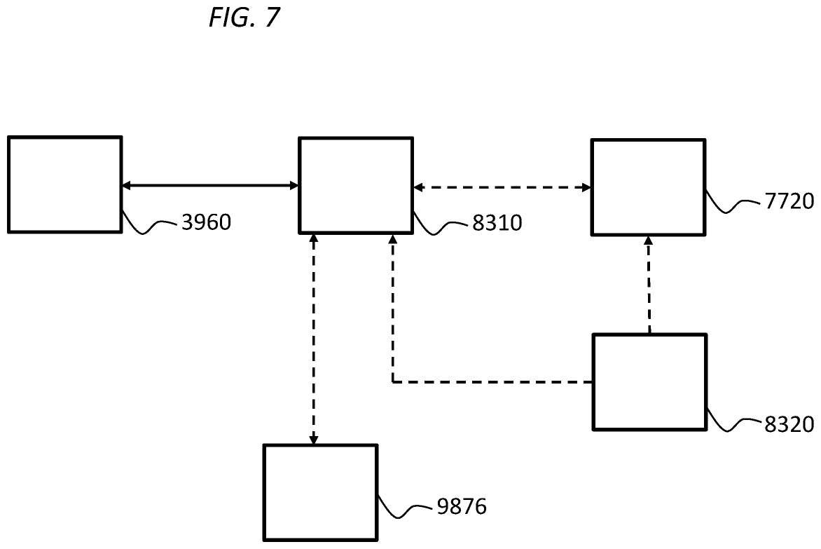

Control unit 8310 can be a signal processor or the like, or a personal computer or the like, or a mainframe computer or the like, etc., that is configured to receive signals from the test unit 3960 and analyze those signals to evaluate the data obtained (it can also be used to control the implant/control the application of current). More particularly, the control unit 8310 can be configured with software or the like to analyze the signals from test unit 3960 in real time and/or in near real time as the electrode array is being advanced into the cochlea by actuator assembly 7720 (if present, and if not present, while the array is being inserted/advanced by hand). The control unit 8310 analyzes the input from test unit 3960, after partial and/or full implantation and/or after the surgery is completed and/or as the electrode array advanced by the actuator assembly 7720 and/or as the electrode array is advanced by the surgeon by hand. The controller/control unit can be programmed to also control the stimulation/control the providing of current to the electrodes during the aforementioned events/situations. The controller 8310 can evaluate the input to determine if there exists a phenomenon according to the teachings detailed herein. The controller can evaluate telemetry, or otherwise receive telemetry, form the implant, via the device that communicates with the implant. That said, in an alternate embodiment, as depicted in FIG. 7, or in addition to this, the controller 8310 can output a signal to an optional monitor 9876 or other output device (e.g., buzzer, light, etc.), that can provide the surgeon or other healthcare professional performing the operation or evaluating the data postoperatively, etc., indicative of the data obtained and/or indicative of a conclusion reached by the control unit 8310. Note also that in an exemplary embodiment, the control unit 8310 can be a dumb unit in the sense that it simply passes along signals to the implant (e.g., the control unit can instead be a series of, for example, buttons where a surgeon depression is one button to provide stimulation to a given electrode). The control unit 8310 can be an external component of the cochlear implant.

Some exemplary embodiments utilize the receiver/stimulator 8710 as a test unit 3910 that enables the action of obtaining the data and the action of providing current to the electrode, and/or any one or more of the method actions detailed herein. In an exemplary embodiment, the receiver/stimulator 8710 and/or control unit 3810 and/or actuator assembly 7720 and/or input device 8320 are variously utilized to execute one or more or all of the method actions detailed herein, alone or in combination with an external component of a cochlear implant, and/or with the interface 7444, which can be used after the receiver/stimulator 8710 is fully implanted in the recipient and the incision to implant such has been closed (e.g., days, weeks, months, or years after the initial implantation surgery). The interface 7444 can be used to control the receiver/stimulator to execute at least some of the method actions detailed herein (while in some other embodiments, the receiver/stimulator can execute such in an autonomous or semi-autonomous manner, without being in communication with an external component) and/or can be used to obtain data from the receiver/stimulator after execution of such method actions.

In some other embodiments, the cochlear implant by itself controls the stimulation and the reading of the data from the read electrodes. In some embodiments, there is a cochlear implant that is configured to autonomously and/or upon instruction or activation by the recipient or other healthcare professional, execute the stimulation and the reading from the read electrodes. In an exemplary embodiment, the data can be stored in the cochlear implant in the memory, and uploaded to a healthcare professional facility or the like (it can be uploaded to system 1206, as will be detailed below, for example) at a utilitarian time in a utilitarian manner. In an exemplary embodiment, there is a cochlear implant that can implement one or more or all of the method actions detailed herein, such as developing the model and/or obtaining the neural response, etc. In an exemplary embodiment, a so-called remote assistant device, such as that embedded in a cell phone or a smart phone or a smart watch or a dedicated electronic component, etc., can be configured to communicate with the hearing prosthesis to implement some or all of the teachings herein. In this regard, any disclosure of any functional or method action herein can be executed in any device disclosed herein providing that the art enables such.

Embodiments include a multi-contact cochlea electrode array, such as those detailed above, an implant with extra-cochlear electrodes (or another component, such as one that works in conjunction with the implanted portion of the cochlear implant), a receiver stimulator (such as that of the implanted portion), which can be either fully implanted or powered by an external behind the ear (BTE) processor or other external device. The implanted portion can include an in-built amplifier configured to measure electrode voltages concurrent to the delivery of electrical current to either the same or adjacent electrode contacts.

Some exemplary utilizations of the embodiments of FIGS. 5-9 will now be described, along with some modifications thereto. Teachings herein can include, embodiments, for example, that correspond to any active implantable device, such as, by way of example only and not by way of limitation, cochlear implants, spinal cord stimulators, pace makers, retinal implants, etc. As will be understood from the above, some embodiments include bio-potential amplifiers for the recording of biologically generated responses to electrical stimulation.

Some embodiments are directed to recording electrically evoked bio-potentials. In some instances, such as those utilizing current technology, there can exist residual artefacts of the electrical stimulation which can be, in some instances, 0.1, 0.2, 0.3, 0.4, 0.5, 0.75, 1, 1.25, 1.5, 1.75, 2, 2.5, 3, 3.5 or 4 or more or any value or range of values therebetween in 0.01 increments (e.g., 0.33, 1.19, 1.28 to 3.37, etc.) orders of magnitude larger than the signal of interest. At least some embodiments, minimize or eliminate this artefact, and can reduce and/or eliminate or otherwise effectively negate or effectively account for any additional noise that results from doing so that results in the measurement procedure.

At least some embodiments include utilizing a mathematical model of the stimulation artefact based on one or more of (i) stimulation parameters, (ii) device configuration (such as the electrode pad size), (iii) device behavioral characteristics, and (iv) interface properties. In at least some embodiments, the model can be used to substantially eliminate the stimulation artefact, without introducing, or at least without introducing or otherwise only introducing minimal additional thermal and/or quantization noise, in addition to potentially providing utilitarian information on the device and the electrode\electrolyte interface.

Some embodiments are based upon the recognition that the stimulation artefact arises predominantly from the electrode-electrolyte interface. For some combinations of biological signals, stimulation paradigms and electrode materials, the stimulation artefact decay rate is substantially different from the biological signal time constant. The present technology can construct mathematical models which adequately model the stimulation artefact, but advantageously retain insufficient flexibility to model the biological signal. This concept is relied upon in at least some embodiments. In this regard, a model may, in some instances at least, if not in all, not be able to model the biological signal. In such a scenario, the biological signal effectively becomes part of the noise in the process. Because the stimulation artefact is usually very large compared to the biological signal of interest, it is expected to have minimal impact on the process.

A brief example will be provided in the context of the Evoked Compound Action Potential (ECAP) response to the stimulus from a cochlear implant. Again, it is noted that in some embodiments, the teachings herein are applicable to other types of response regimes and/or other types of medical devices.

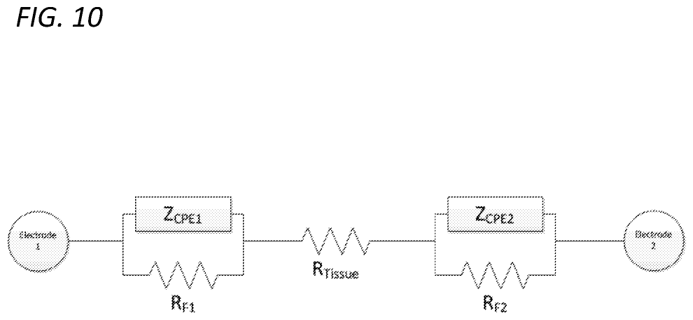

In ECAP, the artefact arising from stimulation requires milliseconds to decay. Conversely, the ECAP biological potential has deviations with a time constant measured in 100's of microseconds. For the purposes of this example, the mathematical model is derived from the Fricke-Warburg model of an electro-tissue interface shown in FIG. 10. Because cochlear implants use platinum electrodes and are thus highly polarizable, the faradaic component will be very large compared to the constant phase element and thus, in some embodiments, may be ignored, so each constant phase element can be defined using two parameters (A and α), by way of example. The interface model can be supplemented with a parameter for the stimulation current source error (Iₑᵣᵣ), restricted to the known device characteristics.

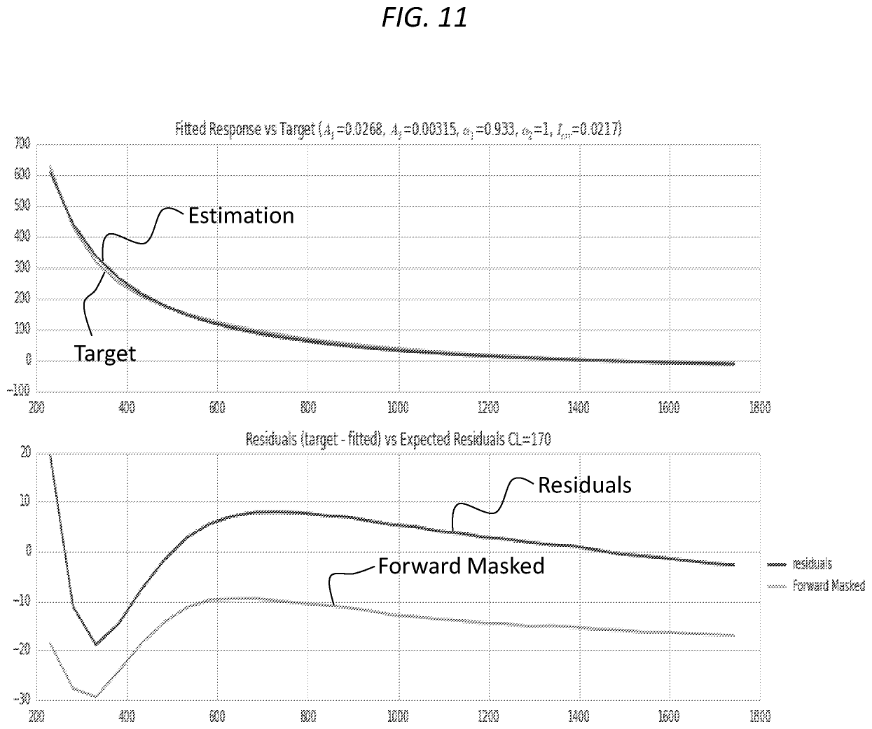

Results are shown in FIG. 11. The upper plot frame shows an average measurement following a 170 CL probe stimulus, from multiple recipients, which is used as the “target” of the model fit. The target is plotted against the estimated stimulation artefact generated by the proposed model. This figure shows just how large the stimulation artefact is with respect to the biological potential, and why it is reasonable to assume fitting using this constrained model will largely be unaffected by the presence of a neural response in the fit. The upper plot represents measurement of probe only stimulus (target) and model of stimulation artefact (estimation).

The lower plot frame in FIG. 11 shows a comparison of a response obtained via forward masking, against the residuals remaining after subtracting the modelled estimation from the target. This can be the calculated neural response (Forward Masked) and response after subtracting the estimation from the target (residual). The present technology results in an ECAP response of similar morphology but with a greater amplitude, which is expected given the inefficiencies of forward masking.

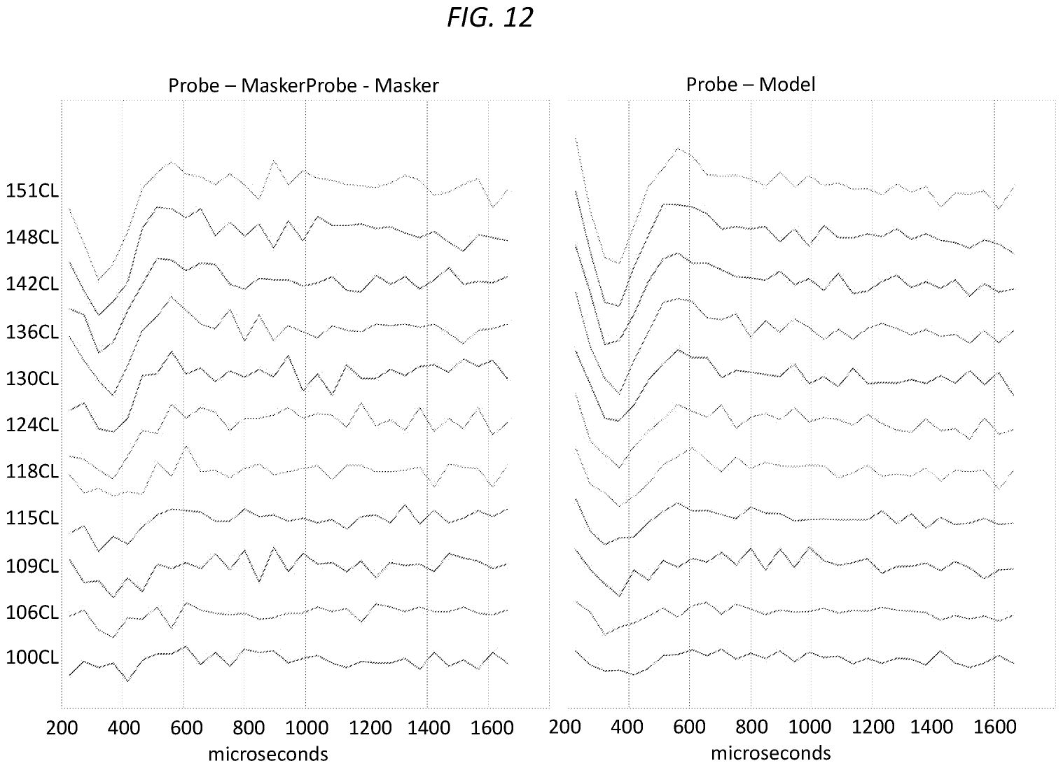

FIG. 12 presents exemplary data associated with taking this fitting technique and applying it to an ECAP measurement set captured using forward masking. More specifically, FIG. 12 represents a comparison of forward masking, to model subtraction of the present technology, using a standard measurement set on a single electrode. With reference to the right side graph, it is clear that the residuals matching the response morphology is not an accident, the responses are generally larger and the noise lower. This technique incorporates the benefit of minimal impact on the biological response morphology without the added time or noise cost.

In an exemplary embodiment, the artefact is treated to be in the form y=mx+d (y being voltage, and x being time), so the model is fitted to measured data according to utilitarian data fitting techniques. In some embodiments (advantageously by design), the artefact model that is being used cannot fit the response (which can be quadratic) irrespective of the size of the intercept. Thus, the original (wanted) biological response can be recovered from the mixed measurement data, by subtracting the artefact model from the measurement data. Thus, in some embodiments, this can be a similar process utilized to develop the initial model for the purpose of artefact fitting. In some embodiments, the shapes of the curves/data plots are substantially different so a model can be constructed which fits the artifact but also (advantageously) cannot fit the response. Notably, because the actual artefact can be a non-linear function, there are drawbacks in trying to model or fit the artefact via simple mean squares. Instead, to allow the use of numerical methods to fit or model the artefact, in some embodiments, a guess is initially taken at the model parameters, with the expectation that the guess is sufficiently close to the final parameters, and then numerical methods are used to refine this guess (repeatedly, in some embodiments) and find the model which results in the smallest error (at least that which has an error that is sufficiently small). This can become the model for the stimulation artifact to be used with the present technology. Because, in some embodiments, the model can be based off physical properties, one can be enabled to make a good guess at the likely parameters (or one can measure some of these parameters using impedance spectroscopy, or any other available method and/or system that can enable such) and use this to seed the fit parameters.

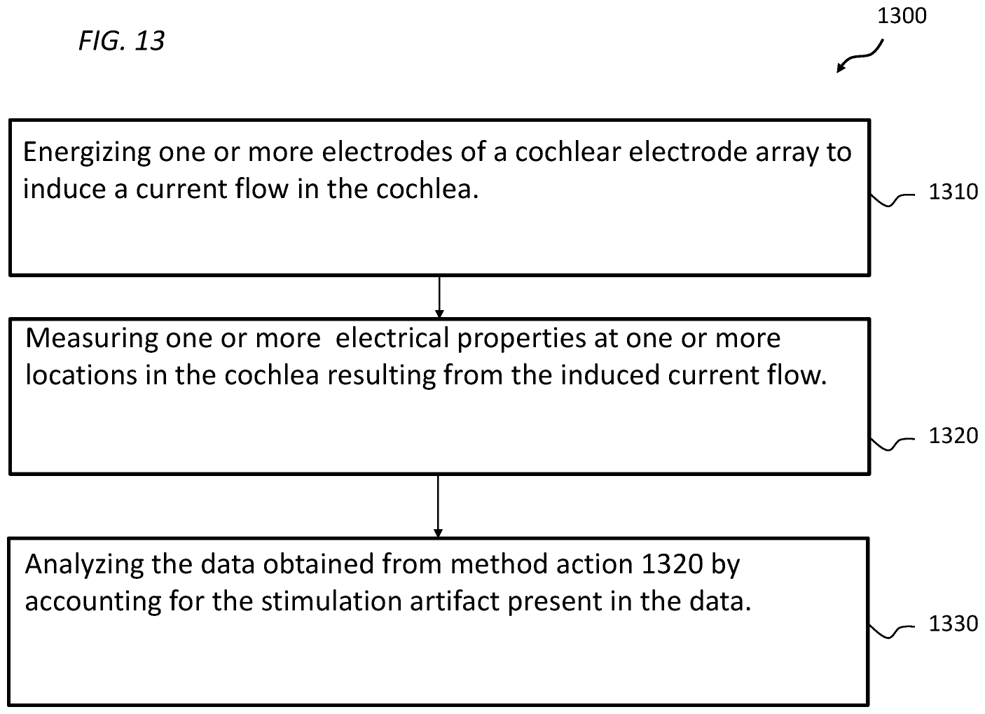

FIG. 13 presents an exemplary high level flowchart for an exemplary method, method 1300, according to an exemplary embodiment. Method 1300 includes method action 1310, which includes energizing one or more electrodes of a cochlear electrode array to induce a current flow in the cochlea. This can be monopolar, bipolar, tripolar stimulation, etc. Any energizement regime that can enable the teachings detailed herein can be utilized in at least some exemplary embodiments.

Method 1300 also includes method action 1420, which includes measuring one or more electrical properties at one or more locations in the cochlea resulting from the induced current flow. In an exemplary embodiment, the measured electrical properties are at different locations along the electrode array after the electrode after the electrode array is fully inserted. That said, some embodiments include executing the method during insertion.

Method 130 also includes method action 1330, which includes analyzing the data obtained from method action 1320 by accounting for the stimulation artefact present in the data. In some embodiments, the result of method action 1330 is to determine whether a neural response signal is included in the data obtained in method action 1320, and, in some embodiments, what exactly makes up that neural response. Techniques to account for the stimulation artefact will now be described.

In an exemplary embodiment, there is the development of an artefact model, which model is used in method action 1330 to analyze the data. In an exemplary embodiment, the development of the model includes fitting the model to the obtained data obtained in method 1320.

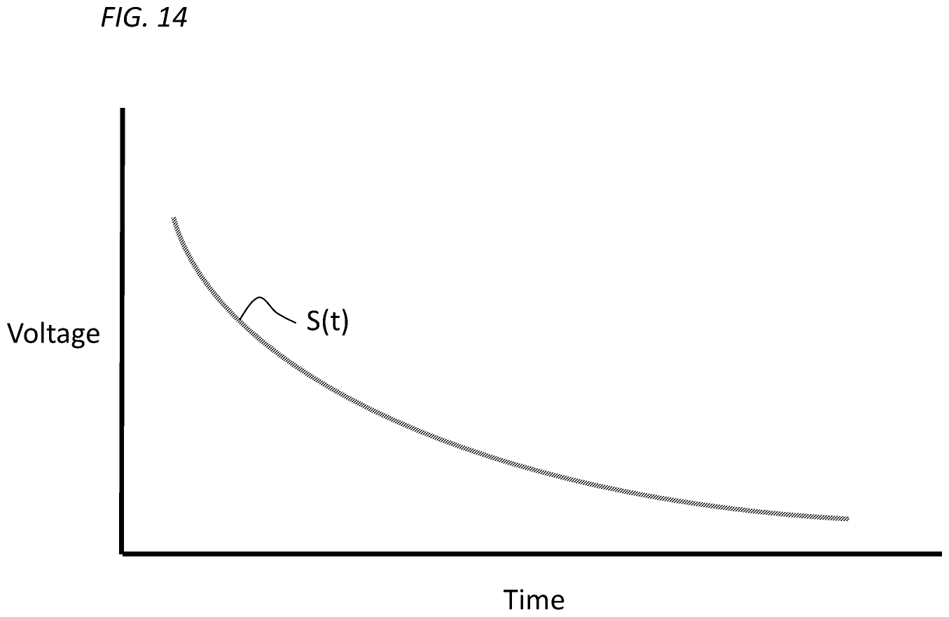

FIG. 14 presents a conceptual voltage vs. time recording obtained via method action 1320. Here, S(t) is the change in voltage with time, and contains the neural response as well as the artefact, which artefact dominates as noted above. That is, the neural response is swamped by the artefact, and thus one can consider FIG. 14 a graph where only the artefact is visible on this scale. (By rough analogy, this is akin to “seeing” the light from distant planets. The star about which the planet orbits dominates, and thus the light from the star is the only light that is visible.)

In the curve of FIG. 14, there exists the signal artefact plus the neural response, random noise. It is noted that at least some exemplary embodiments do not remove the random noise or otherwise account for the random noise that is in the signal while other embodiments do so. The teachings detailed herein are directed towards, in some embodiments, identifying the actual signal artefact and the noise signal, in some embodiments, while in other embodiments, identifying the actual signal artefact, without the noise in the resulting identification.

Accordingly, in at least some exemplary embodiments, the methods disclosed herein can further include the action of ignoring the noise and/or accepting the noise as part of the neural response data. Conversely, some embodiments include methods that further include the action of doing something about the noise, such as trying to remove the noise based on a standard or on assumptions based on the overall system that is utilized to obtain the data.

In at least some exemplary embodiments, the action of obtaining the neural response data and for the method actions that are required to obtain the neural response data is executed without introducing additional thermal and/or quantization noise into the signal and/or resulting data.

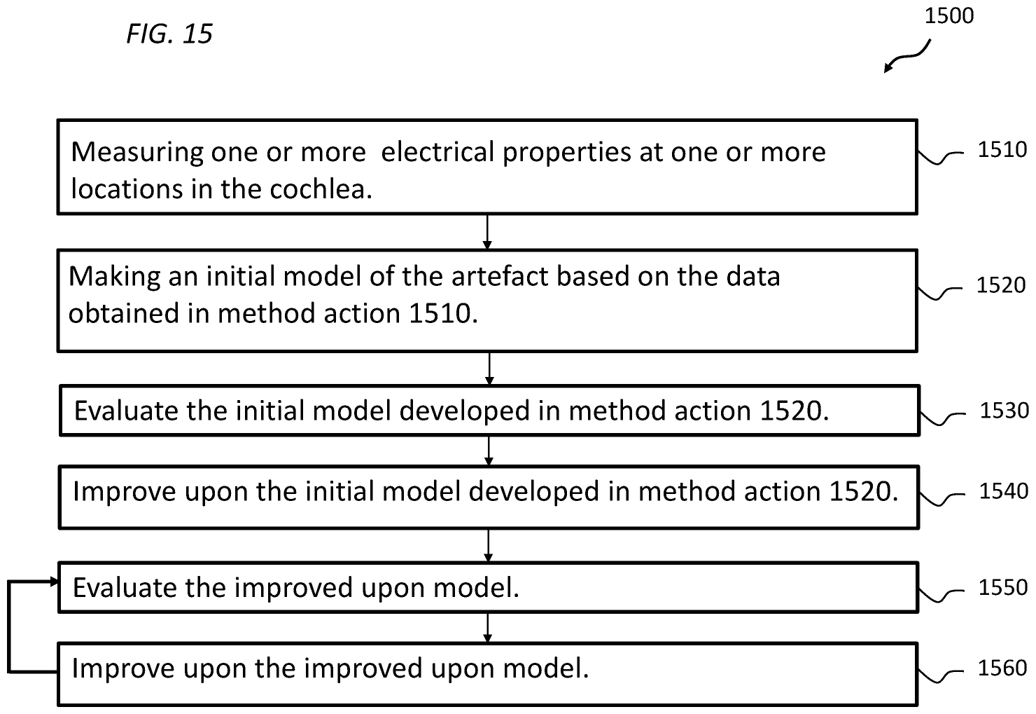

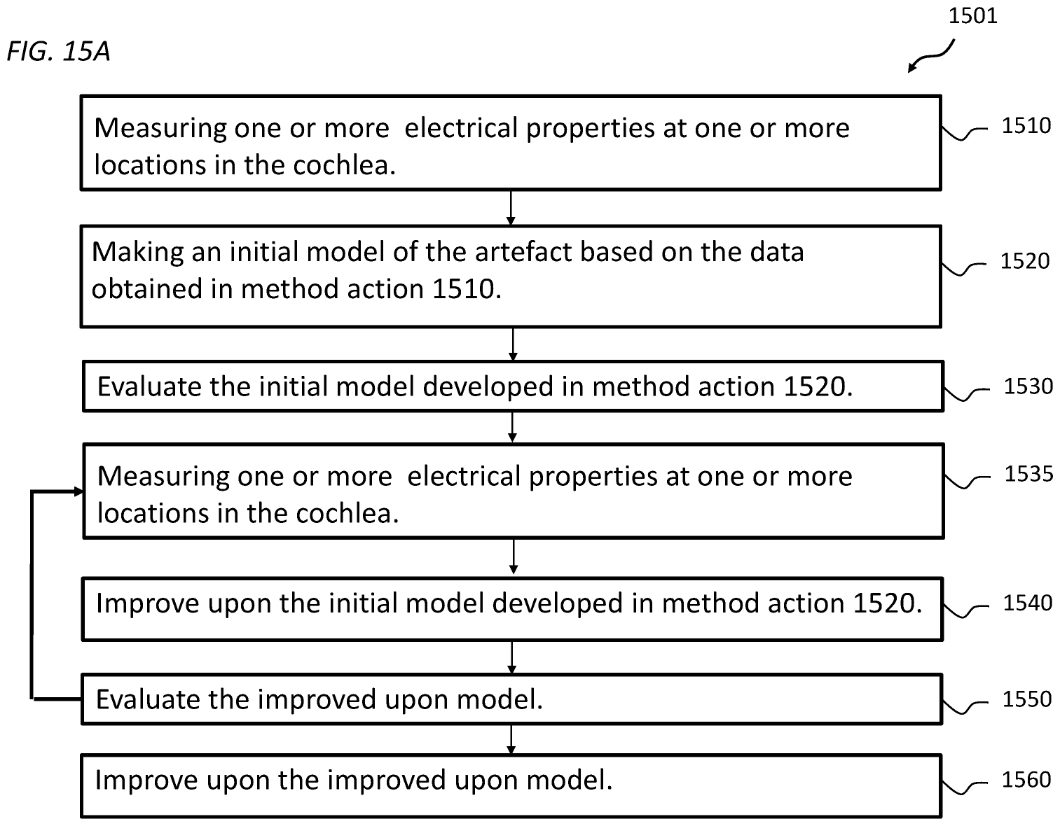

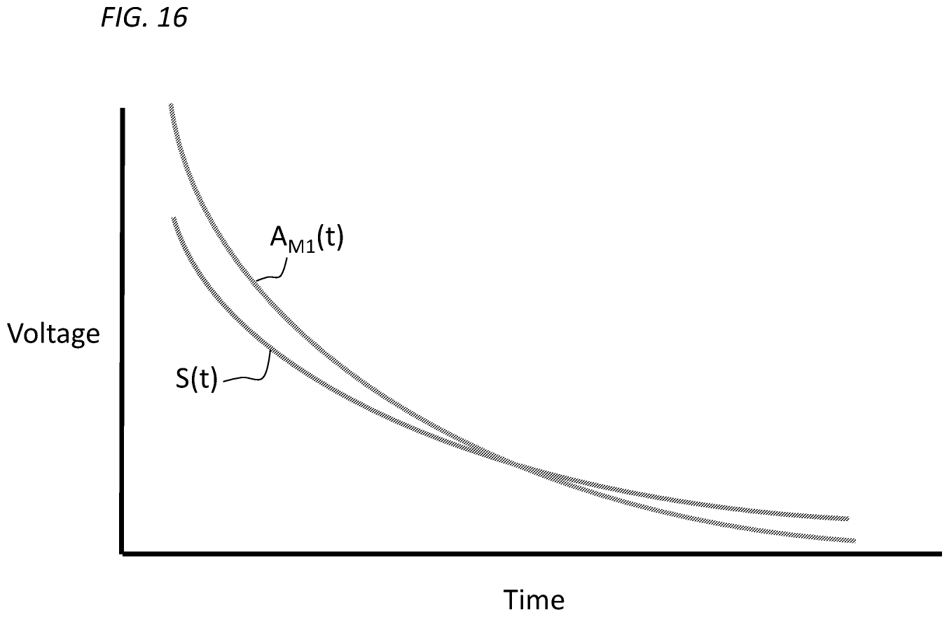

FIG. 15 presents an exemplary flowchart for an exemplary method, method 1500, for developing the artefact model. Method 1500 includes method action 1510, which includes obtaining measurement/signal data, such as that obtained in method action 1420 (method actions 1410 and 1420 can be executed as part of method action 1510. In this embodiment, the data is voltage readings. However, in other embodiments, other electrical properties can be obtained. Any electrical property that can enable the teachings herein can be used in some embodiments.

Method 1500 further includes method action 1520, which includes making an initial model of the artefact based on the data obtained in method action 1510. The initial model can look like curve A_{M1}(t) by way of conceptual example. In an exemplary embodiment, method action 1520 is executed by making one or more guesses for initial values of model parameters and then creating the model response using those parameters. In at least some exemplary embodiments, this initial model may not necessarily be good. However, this is not a problem because the initial model is utilized, in at least some instances, simply to obtain a ballpark concept of how the model should look, from which the model can be further refined.

In this regard, method 1500 further includes method action 1530, which includes evaluating the initial model developed in method action 1520. It is possible that the initial model developed is utilitarian and otherwise can be utilized so that the underlying neural response can be identified in a meaningful or otherwise useful way. If so, no further modeling is executed. That said, in most, if not all scenarios, the initial model developed could be a model that is deemed to be improvable in a meaningful way (more on this below). Accordingly, method 1500 further includes method action 1540, which includes improving upon the initial model developed a method action 1520. Method 1500 further includes method action 1550, which includes evaluating the improved upon model. If it is deemed that the model can be improved upon in a meaningful way, method 1500 then proceeds to method action 1560, which includes improving upon the improved upon model, at which point the method then returned back to method action 1550, which includes evaluating the improved upon model. If it is determined that this second generation of improved upon model can be further improved in a meaningful manner, the method then proceeds to method action 1560, and the cycle is repeated until a determination is made that the improved upon model in a given iteration is utilitarian with respect to implementing the teachings detailed herein to obtain meaningful data related to the neural response. Additional details of this will be described in greater detail below.

FIG. 15A presents an exemplary flowchart for an exemplary method, method 1501, which has some parallels to method 1500 detailed above. In this embodiment, instead of relying on the measurements obtained in method action 1510, measurements are again made after the initial model is developed and/or after subsequent models are developed. In this regard, method 1501 includes method action 1535, which includes measuring one or more electrical properties at one or more locations in the cochlea. It is noted that the locations can be the same as in method action 1510 or can be at different locations. Any locations that can enable the teachings detailed herein can be utilized at least some exemplary embodiments. In any event, it is noted that measurements can be taken repeatedly and/or singularly depending on the utilitarian value associated there with. In an exemplary embodiment, there is a new data set that is obtained for every model that is developed while in other embodiments, there is a new data set that is obtained for every two or three or four or five or six or more models developed, etc.

It is briefly noted that the action of measuring can be located between any of the method actions, as opposed to only those shown in the figure. Indeed, in an exemplary embodiment, it is noted that any the method actions detailed herein can be practiced in any order providing that such can provide utilitarian value and can enable the teachings detailed herein, all unless otherwise noted.

In view of FIG. 15A, it is to be understood that in at least some exemplary embodiments, there are a plurality of data sets that are developed, which data sets can be utilized to further refine and otherwise improve upon the model. Accordingly, any disclosure herein of the utilization of a dataset also corresponds to a disclosure of an embodiment that includes obtaining and/or using two or more datasets. That said, some embodiments utilize only a single dataset.

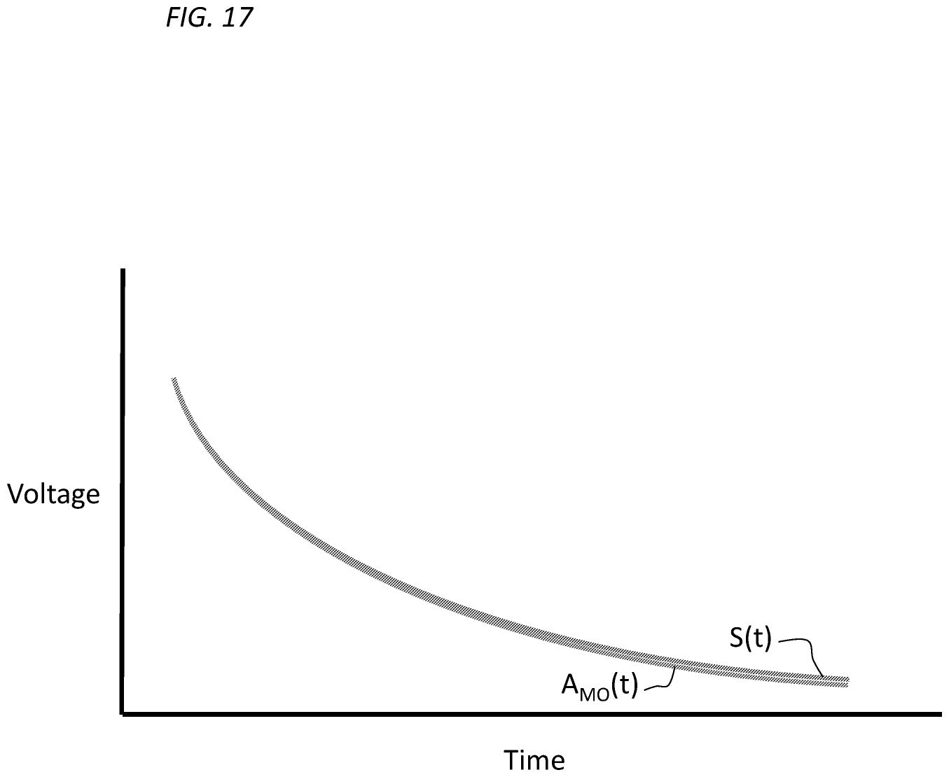

FIG. 17 presents an exemplary embodiment of an improved upon model, represented by curve A_{MO}(t). In an exemplary embodiment, this can be the second, third, fourth, fifth, sixth, seventh, eighth, ninth, tenth, 11^{th}, 12^{th}, 13^{th}, and so on iteration of the model. With respect to the iterations of the model, where n equals the first iteration developed at method action 1520, n can be any integer from 1 to 10,000 or higher or any value or range of values therebetween in one increment (e.g., n can be 5, 8, 10, 3-33, 8 to 134, etc.). Still, to be clear, at some point, the iterations begin to yield diminishing returns, so thus the larger number may not be experienced in most instances.

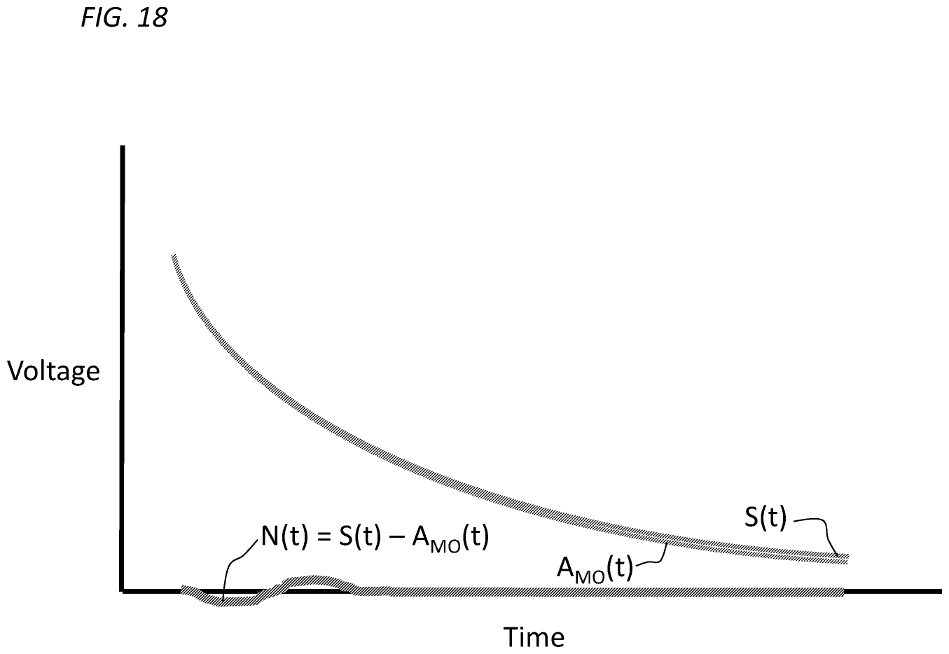

Returning back to method action 1330, the action of analyzing the data obtained from method action 1320 by accounting for the stimulation artefact present in the data can be executed in a manner represented by way of example only and not by way of limitation, by FIG. 18. In an exemplary embodiment, the neural response, N(t), can be found by the equation N(t)=S(t)−A_{MO}(t). Thus, method action 1330 can be executed once a utilitarian model, which can be the, optimum model, for the artefact, A_{MO}(t) is obtained, by subtracting that model from the original signal, S(t), obtained in method action 1320, to obtain neural signal N(t). FIG. 18 conceptually demonstrates that, usually, N(t) is very small compared to the artefact and often cannot be seen on a plot of S(t).

In an exemplary embodiment, the iterations of the models change from iteration to iteration so that the models begin to converge on the curve for the measurement, but never fully converges. The models cannot fully converge because if such is the case, it would not be possible to extract the neural response from the data. Accordingly, in at least some exemplary embodiments, the goal is to develop a model that is good enough or close enough, and then stopping.

Briefly, it is noted that, with respect to FIG. 18, it can be seen that the artefact model begins to diverge from the signal data with time, and that divergence increases with time. This is an occurrence that exist in at least some exemplary embodiments. This is because the neural response decays faster than the artefact, in at least some exemplary embodiments. At least some exemplary embodiments rely on this phenomenon to develop the model or otherwise to distinguish from the signal data.

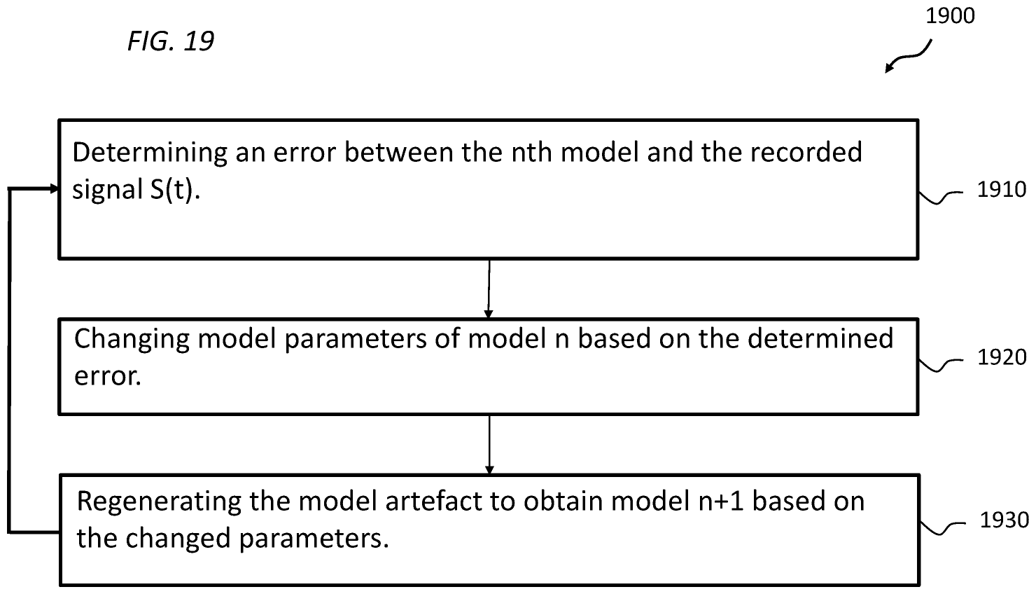

FIG. 19 presents an exemplary flowchart for an exemplary method, method 1900, for improving the artefact model. Method 1900 includes method action 1910, which includes determining an error between the n^{th} model and the recorded signal S(t). Based on the determined error, which can be determined by calculating the error between the n^{th} model and the recorded signal S(t), model parameters are changed in method action 1920.

Method 1900 further includes method action 1930, which includes regenerating the model artefact to obtain the n+1^{th} model, which in this embodiment, where n equals 2, would be A_{M2}(t). Method 1900 then returns to method action 1910, where the process is repeated as many times until a determination is made, for example, as a result of method action 1910, that the error determined between the nth model and the recorded signal is sufficiently low that the model can be utilized in a utilitarian manner to determine the neural response/that the error is sufficiently low that the model utilized will provide a utilitarian neural response value.

Thus, it can be seen that method action 1900 is repeated n−1 times, generating artefact models A_{Mn}(t) every time, until a model is developed that is deemed satisfactory.

Eventually the best or optimum model is found, such as, for example, when the error cannot be decreased further, or at least decreased further in a meaningful way (there are many methods or algorithms for changing the model parameters in response to the calculated error which can be utilized—any error analysis regime can be utilized to enable the teachings detailed herein can be utilized in at least some exemplary embodiments). In an exemplary embodiment, upon a determination that the overall error has not decreased by more than 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, or 0.001% or less, or any value or range of values therebetween in 0.001% increments, for 1, 2, 3, 4, 5, 6, 7, 8, 9, 10, 11, 12, 13, 14, 15, 16, 17, 18, 19, 20, 25, 30, 35, 40, 45, or 50 or more, or any value or range of values therebetween in one increment iterations between models (adjacent otherwise), the latest model or one of those models meeting the above-noted criteria can be utilized as the model that will be subtracted or otherwise utilized to remove the artefact from the measured signal.

In an exemplary embodiment, the final artefact model that is utilized is model A_{MO}(t).

It is noted that in at least some embodiments, there is never a perfect fit between the original signal and the artefact model (e.g., error reduced to zero). Some embodiments are directed to avoiding such an occurrence. Indeed, if the artefact model was a perfect fit to the original signal then subtracting the two would yield nothing, and thus the neural response would not be identifiable.

In some embodiments, the artefact models are developed by purposely constraining the artefact model to take the form of a constant phase element (CPE). In an exemplary embodiment, this is done because the shape of the artefact described sufficiently well by a CPE. Conversely, in some embodiments, method actions 1310 and 1320 are executed such that the neural signal that results therefrom is at least effectively or statistically nothing like a CPE. The neural signal of method actions 1310 and 1320 can be somewhat akin to a damped sinusoid. That is, in at least some exemplary body, the action of generating electrical current and/or the action of measuring the resulting electrical properties within the cochlea are executed in a manner that the underlying neural response is as just described, and thus, if a neural signal is present, the model will never be a perfect fit because it is forced to take the form of a CPE. Accordingly, the teachings detailed herein provide, in at least some exemplary embodiments, avoidance of a scenario where the neural signal is “modelled out.” Accordingly, at least some exemplary embodiments provide guarantee that if there is a neural signal present, the neural signal will always show off when the artefact model is removed from the recorded signal.

Briefly, it is noted that the description above refers to and works from a single waveform. However, in at least some exemplary embodiments, the processes detailed above are applied to a series of waveforms. By way of example only and not by way of limitation, different current levels may be used to record each waveform in the series. In such an exemplary embodiment, a series of artefact models are generated, the models respectively likely using the same or related parameters relative to each other. For example, the parameter that scales the overall amplitude of the artefact model may scale linearly with the stimulation current. The model improvement process then calculates the errors for all the waveforms, sums them, and finds the improved parameters which can minimize the summed errors for all the waveforms. In at least some exemplary embodiments, this is more efficient than repeating the process for each individual waveform because the optimum parameter set for one waveform may be the same or very similar to that for the other waveforms in the series.

Some additional details of developing the CPE based model will now be described.

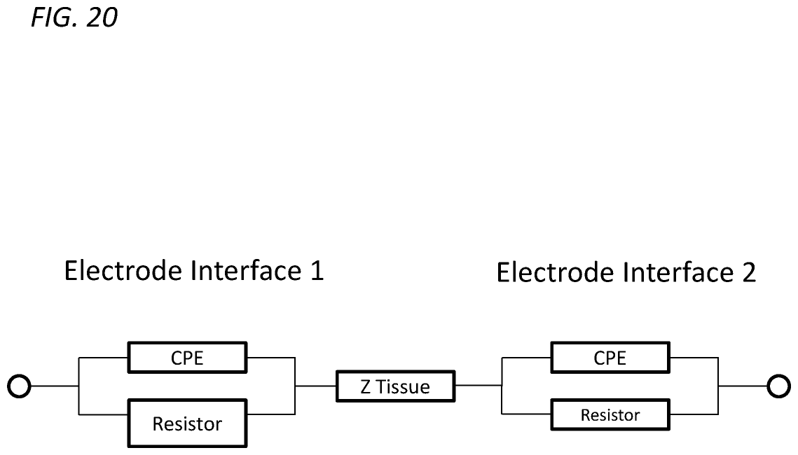

FIG. 20 presents an exemplary conceptual schematic representing electrode interface interaction of the electrodes utilized in the cochlear implants electrode array that is utilized to implement method actions 1310 and 1320. The model represented by the schematic of FIG. 20 can be represented via a CPE based time equation as follows:

$${V}_{τ} ( t ) = [ \frac{{( t - τ )}^{α}}{A Γ ( α + 1 )} ] {I}_{τ} u ( t - τ )$$

### Where:

I: Stimulation current (Amps)
t: Time (Seconds)
τ: Time offset (Seconds)
A: double layer magnitude (1/ohms) (All things being equal this will be proportional to the electrode cross sectional area)
α: Frequency sensitivity factor (eg, 0=Resistor [i.e. no frequency sensitivity] and 1=Capacitor)
Γ: Gamma function
u(t): Heavyside step function

The units of “A” can be Siemens×seconds^{alpha} or 1/omega×seconds^{alpha} (because Farads=S/omega, so when alpha is 1 it will have units in Farads, when it is zero one has units in 1/omega or admittance), the units of S can be J×radians/second, and omega equals resistance. In some instances, alpha thus becomes unitless and effectively a frequency dependent factor.

In some embodiments, such as for a cochlear implant electrode array, the value for A can be around or be actually 10⁻⁶. This is based on the fact that equivalent capacitance of an intracochlear electrode measured at around 10⁴ radians per second is around 10⁻⁸ Farads. That entails treating it like a capacitor where alpha=1. Also, the same electrode, if thought of as a conductance (=1/resistance), has a conductance of around 10⁻⁴ S or 10⁻⁴ Ohms⁻¹. That can be based on an assumption that alpha=0. So assuming alpha is typically 0.5, the value of A that gives the equivalent admittance (=1/impedance) will be 10⁻⁶ in at least some instances. If alpha is closer to 1 (the interface behaves more like a capacitor), then the value for A will be closer to 10⁻⁸. If alpha is closer to 0 (the interface behaves more like a resistor), then the value for A will be closer to 10⁻⁴. In practice alpha can vary quite a bit and thus the value of A can also vary a lot (for example, 10⁻⁵ to 10⁻⁷, by way of example).

Also, alpha (α) works out to around 0.5 as taken from empirical measurements of platinum electrodes in a saline solution (although the range is around 0.3 to 0.7).

In some embodiments, it can be assumed that R is large enough such that the impact thereof to the model can be considered not to matter, the above equation can be expanded to establish an equation for the full model as follows:

$${V}_{τ} ( t ) = [ \frac{{( t - τ )}^{{α}_{1}}}{{A}_{1} Γ ( {α}_{1} + 1 )} ] {I}_{τ} u ( t - τ ) + {Z}_{Tissue} {I}_{τ} u ( t - τ ) + [ \frac{{( t - τ )}^{{α}_{2}}}{{A}_{2} Γ ( {α}_{2} + 1 )} ] {I}_{τ} u ( t - τ )$$

### Where:

I: Stimulation current (Amps)
t: Time (Seconds)
τ: Time offset (Seconds)
A: double layer magnitude (1/ohms) (All things being equal this will be proportional to the electrode cross sectional area)
α: Frequency sensitivity factor (0=Resistor [i.e. no frequency sensitivity] 1=Capacitor
Γ: Gamma function
u(t): Heavyside step function

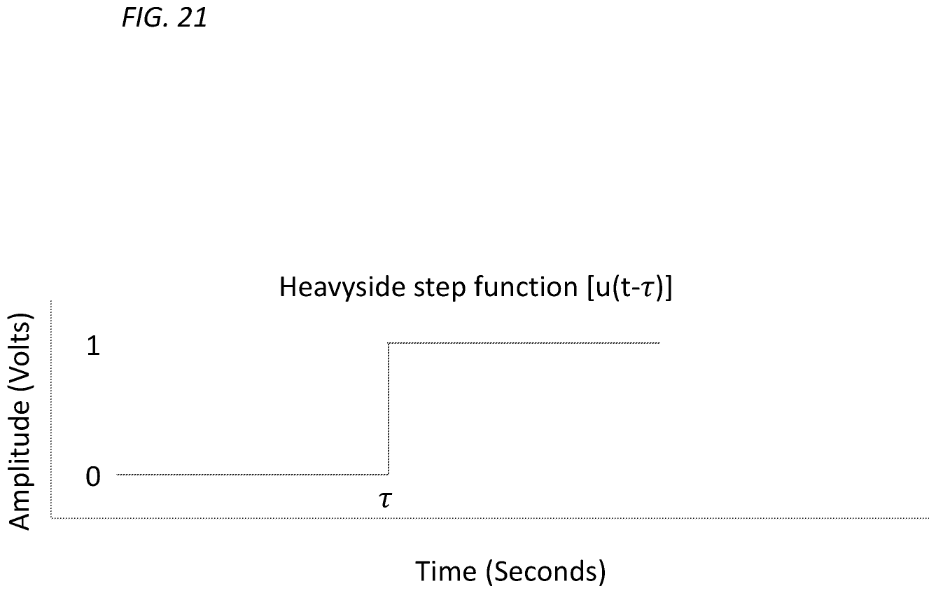

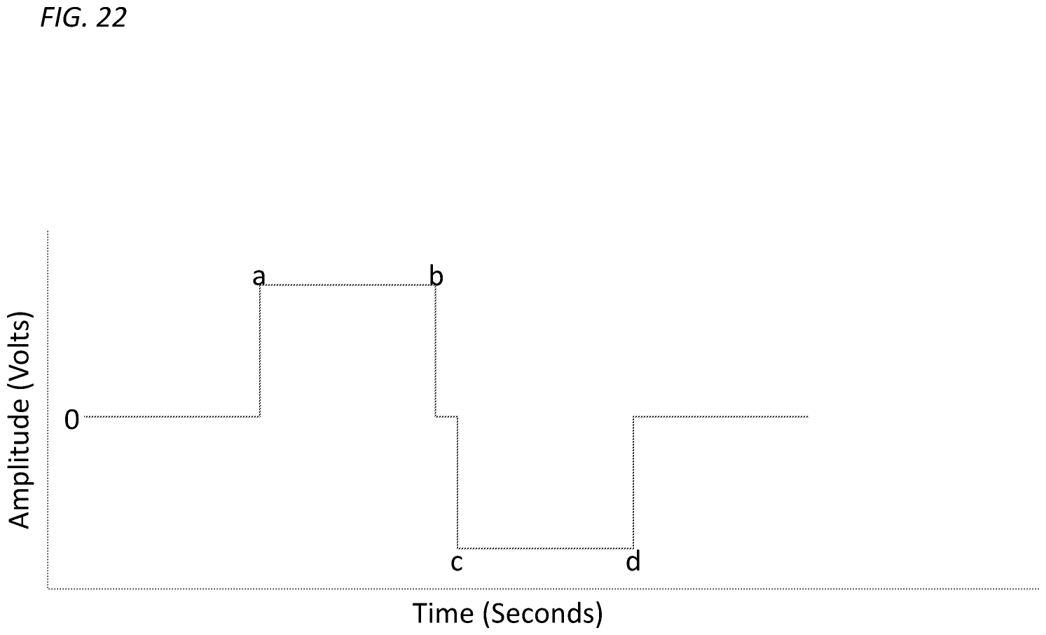

FIG. 21 schematically represents a heavyside step function, and FIG. 22 schematically represents that a biphasic stimulus can be constructed from four heavy side step functions at times a, b, c & d as shown. The values of the artefacts for the biphasic stimulus can be calculated using the following equations:

$${V}_{Artifact} ( t ) = {V}_{a} ( t ) - {V}_{b} ( t ) - {V}_{c} ( t ) + {V}_{d} ( t )$$

$${V}_{Artifact} ( t ) = [ \frac{{( t - a )}^{α}}{A Γ ( α + 1 )} ] {I}_{τ} u ( t - a ) - [ \frac{{( t - b )}^{α}}{A Γ ( α + 1 )} ] {I}_{τ} u ( t - b ) - [ \frac{{( t - c )}^{α}}{A Γ ( α + 1 )} ] {I}_{τ} u ( t - c ) + [ \frac{{( t - d )}^{α}}{A Γ ( α + 1 )} ] {I}_{τ} u ( t - d )$$

The full model would comprise, in some embodiments, two CPE models that would be fitted.

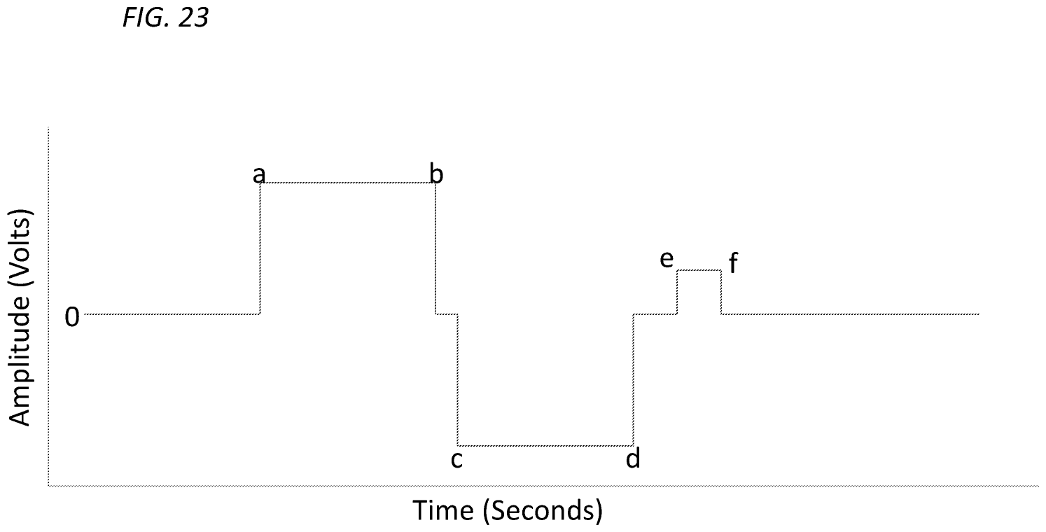

FIG. 23 presents a schematic representing tri-phasic stimulus, and the below equations can be utilized to calculate the values of the artefacts for such:

$${V}_{Artifact} ( t ) = {V}_{a} ( t ) - {V}_{b} ( t ) - {V}_{c} ( t ) + {V}_{d} ( t ) + {V}_{e} ( t ) - {V}_{f} ( t )$$

$${V}_{Artifact} ( t ) = [ \frac{{( t - a )}^{α}}{A Γ ( α + 1 )} ] {I}_{τ} u ( t - a ) - [ \frac{{( t - b )}^{α}}{A Γ ( α + 1 )} ] {I}_{τ} u ( t - b ) - [ \frac{{( t - c )}^{α}}{A Γ ( α + 1 )} ] {I}_{τ} u ( t - c ) + [ \frac{{( t - d )}^{α}}{A Γ ( α + 1 )} ] {I}_{τ} u ( t - d ) + [ \frac{{( t - e )}^{α}}{A Γ ( α + 1 )} ] {I}_{τ} ( t - e ) - [ \frac{{( t - f )}^{α}}{A Γ ( α + 1 )} ] {I}_{τ} u ( t - f )$$

In some embodiments, for triphasic stimulus, allowing for a different value of alpha for Ve and Vf could result in more utilitarian fits.

It is noted that while the above equations are presented in the time domain, this can be done in the frequency domain as well.

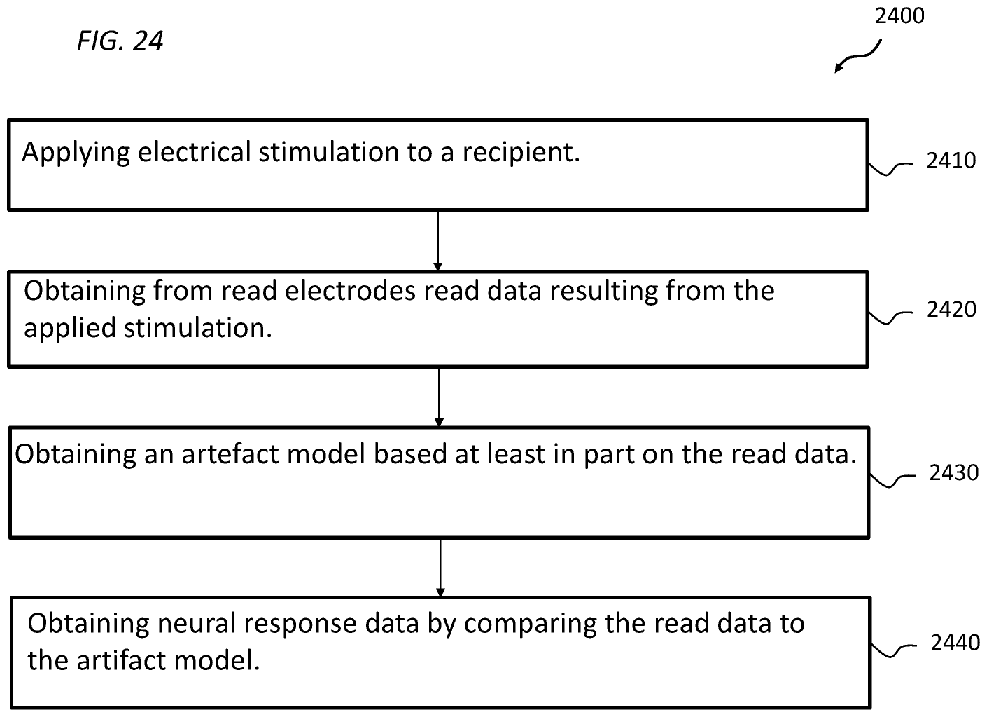

FIG. 24 presents a flowchart for an exemplary method, method 2400, which includes method 2410, which includes applying electrical stimulation to a recipient, such as via a cochlear implant electrode array, or any other arrangement, implanted or otherwise. Method 2400 further includes method action 2420, which includes the action of obtaining from read electrodes read data resulting from the applied stimulation. The read data can be in one or more datasets as detailed above. These read electrodes can be the read electrodes of the cochlear implant electrode array in an embodiment where such is utilized to implement method 2400, or any other read electrodes that can be utilized according to any of the teachings detailed herein. Method 2400 further includes method action 2430, which includes obtaining an artefact model based at least in part on the read data. This method action can be executed according to the teachings detailed herein or any other teaching that can have utilitarian value. Method 2400 further includes method action 2440, which includes obtaining neural response data by comparing the read data to the artefact model. In the embodiments described above, the artefact model is subtracted from the read data. That said, other data manipulation techniques can be utilized aside from or in addition to subtraction. By way of example only and not by way of limitation, least mean squares analysis could be utilized or any other statistical analysis could be utilized, providing that such results in utilitarian results. Any data manipulation techniques that can be utilized to execute the comparison between the artefact model and the read data can be utilized in at least some exemplary embodiments.

In an exemplary embodiment, as will be understood from the above, the actions of applying and obtaining are part of an eCAP measurement method (an electrically evoked compound action potential measurement method). Thus, in an exemplary embodiment, the application of electrical stimulation and the obtaining of the read data occurs at a cochlea of a person. It is noted that the teachings herein are not limited to eCAP. Any measurement regime where artefacts are an issue can be a measurement regime to which the teachings herein can be applied.

As noted above, the constant phase element analysis that is utilized to develop the model can, in some instances, rely on pre-determined or otherwise pre-known initial parameters (which parameters can be assumptions based on empirical or analytical efforts, or can be exacting parameters—any parameters that can enable the teachings detailed herein can be utilized in at least some embodiments. Thus, in an exemplary embodiment, the stimulation applied to the recipient meets certain parameters and the obtained artefact model is based on the certain parameters and based on the read data. In some embodiments, as noted above, stimulation parameters (step function, bipolar, tripolar, etc.), device configuration parameters, such as for example, the electrode pads size or geometry, etc., device behavioral characteristics that can be parameterized, and/or tissue interface property parameters can be utilized in at least some exemplary embodiments.

In a sense, the parameters that are utilized can be considered “seed parameters” which can be utilized to develop “seed parameter estimates” for use in the models, such as to develop the values for the equations detailed above. The key here is that by utilizing devices systems and methods that harness standard parameters, or at least known parameters, the constant phase element-based equations can be developed in a manner that can yield a utilitarian artefact model.

In some embodiments, the artefact model according to the teachings detailed herein is an artefact model that is based on a true constant phase model. In some embodiments, the artefact model does not rely on the results of a double exponential.

As can be seen from the above, the model improvement actions, such as those detailed in method 1900 above, result in an artefact model that is specific to an exact recipient. This as opposed to a model that is based on statistical data for a classification of recipients, etc. accordingly, in an exemplary embodiment, there is a method that comprises developing a recipient-specific electrical stimulation artefact model. In an exemplary embodiment, the developed stimulation artefact model is developed by using predetermined constants and by using data from in-situ electrodes. As noted above, the model can be based on a constant phase model.

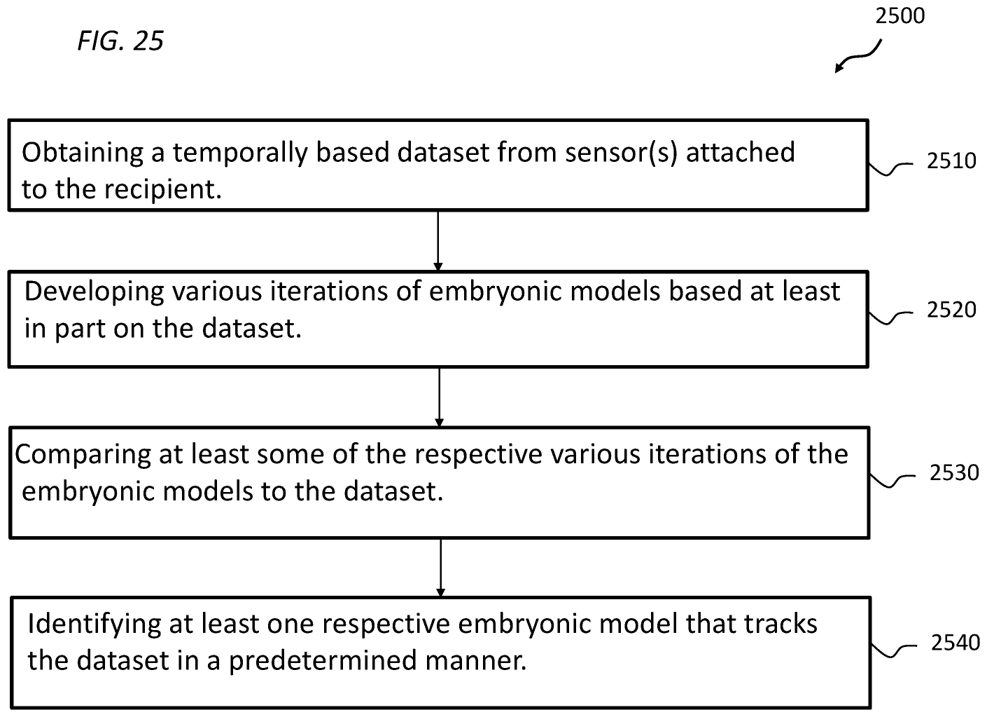

FIG. 25 presents a flowchart for an exemplary method, method 2500, for the development of the recipient-specific model. Method 2500 includes method action 2510, which includes obtaining a temporally and/or frequency based dataset from sensor(s) attached to the recipient. This can be done utilizing the read electrodes of the cochlear implant electrode array, the read electrodes of a pacemaker, the electrodes of a retinal implant, the electrodes of a brain stimulator, etc. Method 2500 further includes method action 2520, which includes developing various iterations of embryonic models based at least in part on the dataset obtained in method action 2510, and method action 2530, which includes comparing at least some of the respective various iterations of the embryonic models to the dataset. Here, the embryonic model refers to a model that is developed but has not yet reached the stage of a full model that has been deemed sufficient to be used as the artefact model. This can correspond to one or more of the nth models detailed above. In this regard, method 2500 includes method action 2540, which includes identifying at least one respective embryonic model that tracks the dataset in a predetermined manner (e.g., the error difference is within a given range, etc.). In an exemplary embodiment of method 2500, the model is based at least in part on the identified at least one respective embryonic model identified in method action 2540. In an embodiment, the identified at least one respective embryonic model is the model (becomes the model). In an embodiment, a plurality of respective embryonic models that track the data set in a predetermined manner are average or otherwise statistically manipulated to arrive at model. In both instances, the model is based at least in part on the identified at least one respective embryonic model identified in method action 2540. Any regime that can be utilized in a utilitarian manner that can develop the model based on the embryonic models can be utilized in at least some exemplary embodiments.

It is briefly noted that in an alternate embodiment (it is noted that the embodiment of FIG. 25 does not exclude this), the very first model can be based on a population mean or some other statistically significant data set, results of a previous fitting session with the recipient who is the subject of the method of FIG. 25, and or alternative the measurements performed via the same electrode configuration (for example, spectroscopy, impedance/transimpedance). In an exemplary embodiment, there can be a temporal difference between the development of the first model and the development of the second model, or at least the actions of obtaining the underlying data utilized to develop the very first model relative to the subsequent models, of at least 1, 2, 3, 4, 5, 6, 7, 8, 9, 10, 11, 12, 13, 14, 15, 20, 25, 30, 35, 40, 45, 50 or more days or weeks or any value or range of values therebetween in 1 day or week increments. Accordingly, in an exemplary embodiment, the very first model can be developed based on data that is not obtained contemporaneously with the data that is utilized to develop the subsequent models. That said, in an alternative embodiment, the models are developed utilizing one data set, which data set is obtained at the beginning of the method.

It is also briefly noted that while the embodiments herein often focus on temporally based data sets, in alternate embodiments, the teachings herein can be implemented based on frequency based data sets. Any disclosure herein of a temporally based data set corresponds to a disclosure of an alternate embodiment of a frequency based data set (or at least obtaining and/or utilizing one) and vice versa unless otherwise specifically noted. That is, in embodiments herein can be executed utilizing the time domain and/or the frequency domain data.

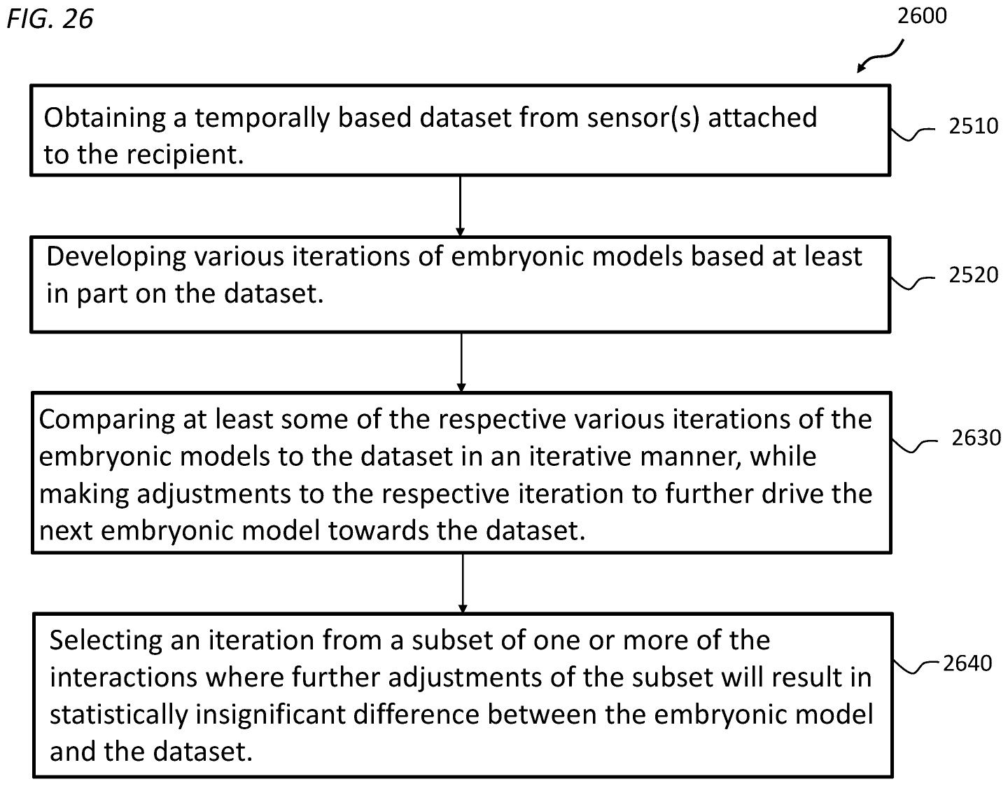

FIG. 26 presents another exemplary algorithm for an exemplary method, method 2600, that includes method actions 2510 and 2520. Method 2600 also includes method action 2630, which includes comparing at least some of the respective various iterations of the embryonic models to the dataset in an iterative manner, while making adjustments to the respective iteration to further drive the next embryonic model towards the dataset. This is done in accordance with the teachings herein, in some embodiments. Method 2600 also includes method action 2640, which includes selecting an iteration of the embryonic models from a subset of one or more of the iterations of embryonic models where further adjustments of the subset will result in a statistically insignificant difference between the iteration of the embryonic model and the dataset.

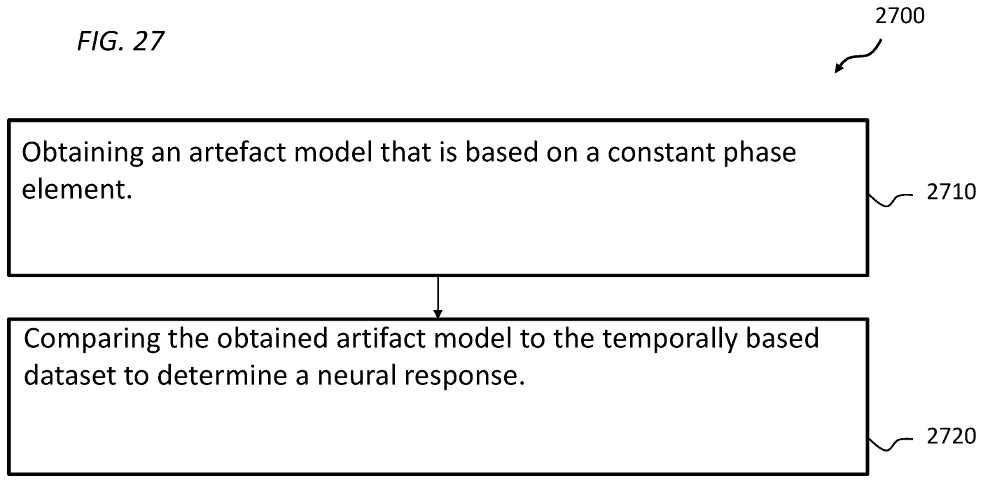

In an exemplary embodiment of at least some of the methods herein, the action of developing the model includes obtaining a temporally based dataset from sensors attached to the recipient (which includes implanted in the recipient), developing various iterations of embryonic models, all of which are intended to be different from the dataset and using one of the iterations as a basis for the model (in some embodiments, the method includes using one of the iterations as the model, as noted above). As detailed above, the models are purposely designed to be different then the data set that is obtained from the sensors so as to enable a comparison between the two to develop the actual neural response data. In this regard, FIG. 27 provides an exemplary algorithm for an exemplary method, method 2700, which includes method action 2710, which includes obtaining an artefact model that is based on a constant phase element, which action of obtaining can be executed in accordance with any of the teachings detailed herein or any other method that can enable the teachings detailed herein. Method 2700 further includes method action 2720, which includes comparing the obtained artefact model to the temporally based dataset to determine a neural response. Accordingly, in an exemplary embodiment, the iteration selected the method action 2640 can be utilized in method action 2720 as the model that is compared to the dataset in that action. Still further, in an exemplary embodiment, the respective embryonic model that is identified in method action 2540 can be the model that is compared to the data set in method action 2720 or can be a model that the ultimate model that is utilized in method action 2720 is based upon.

In an exemplary embodiment, method action 2720 is executed by utilizing one or more of the iterations individually and/or collectively (by collectively, the models can be averaged, etc.) to compare to the temporally based dataset to determine a neural response based on the comparison.

In view of the above, it can be seen that the actions of developing the model can include obtaining a temporally based dataset from sensors attached to (including implanted in) a recipient and developing the model at least in part based on the obtained dataset. In some embodiments, the method further comprises comparing this developed model, which was developed based on the dataset, to the dataset to identify a neural response.

It is noted that in at least some exemplary embodiments, the teachings detailed herein are directed to artefact suppression and/or elimination and/or artefact accounting techniques utilized in neural response telemetry (NRT). In an exemplary embodiment the teachings detailed herein can provide faster and/or softer NRT relative to that which would be the case if other techniques, such as those detailed below, are utilized. In an exemplary embodiment, this can be because one does not need to utilize more time-consuming techniques and/or one does not need such large signals to deal with imperfections in the artefact suppression and/or the artefact accounting, all other things being equal (note that any comparisons detailed herein are comparisons made, in at least some exemplary embodiments, under the regime of all other things being equal).

At least some exemplary embodiments of the teachings detailed herein provide the best model for an NRT artefact as of Apr. 1, 2019, with respect to those publicly known or utilized in the United States, Canada, the European Union, the United Kingdom, France, Germany, Australia, New Zealand, China, Japan, and/or India. In some embodiments, the just detailed comparison is with respect to methods and systems that are licensed for use in any one or more of the just mentioned jurisdictions as of the just mentioned date, such as, for example, licensed and/or approved by the Food and Drug Administration of the United States of America on Apr. 1, 2019.

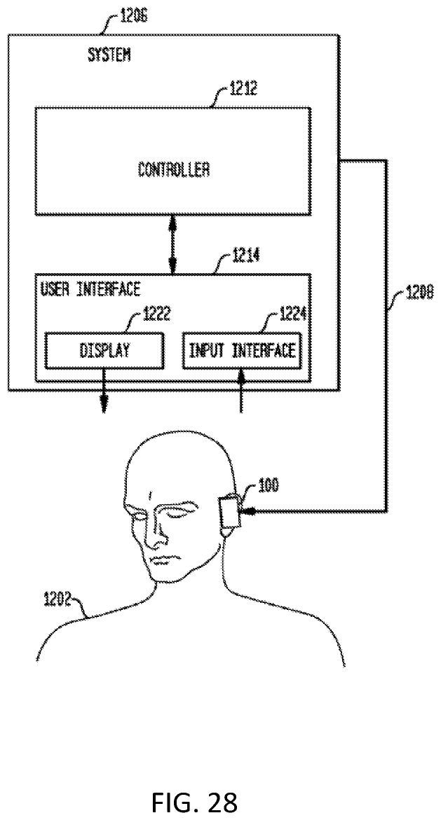

In an exemplary embodiment, there is an electrical response stimulation measurement system having functionality according to the method actions detailed herein. In the embodiment illustrated in FIG. 28, the implant is placed into communication with system 1206, such as, via device 7444, or, for example, via the external component of the overall hearing prosthesis (represented by element 100 in FIG. 28), or a modified device 7444 used for external communication (indeed, device 7444 can be used extracutaneously for that matter, in some embodiments), thus establishing a data communication link 1208 between the hearing prosthesis 100 (where hearing prosthesis 100 is a proxy for any device that can enable the teachings detailed herein) and system 1206. System 1206 is thereafter bi-directionally coupled by data communication link 1208 with hearing prosthesis 100 (or particular part thereof, such as the implant—element 100 is a proxy for any device that can enable the teachings herein that interfaces with the recipient). Any communications link that will enable the teachings detailed herein that will communicably couple the implant and system can be utilized in at least some embodiments.

System 1206 can comprise a system controller 1212 as well as a user interface 1214. Controller 1212 can be any type of device capable of executing instructions such as, for example, a general or special purpose computer, a handheld computer (e.g., personal digital assistant (PDA)), digital electronic circuitry, integrated circuitry, specially designed ASICs (application specific integrated circuits), firmware, software, and/or combinations thereof. As will be detailed below, in an exemplary embodiment, controller 1212 is a processor. Controller 1212 can further comprise an interface for establishing the data communications link 1208 with the hearing prosthesis 100 (again, which is a proxy for any device that can enable the methods herein—any device with a microphone and/or with an input suite that permits the input data for the methods herein to be captured). In embodiments in which controller 1212 comprises a computer, this interface may be, for example, internal or external to the computer. For example, in an exemplary embodiment, controller 1206 and cochlear implant may each comprise a USB, FireWire, Bluetooth, Wi-Fi, or other communications interface through which data communications link 1208 may be established. Controller 1212 can further comprise a storage device for use in storing information. This storage device can be, for example, volatile or non-volatile storage, such as, for example, random access memory, solid state storage, magnetic storage, holographic storage, etc.

In an exemplary embodiment, input is provided into system 1206 from the implant, which input can correspond to the measurements detailed herein. In an embodiment, the system is configured to execute one or more or all of the method actions detailed herein, or at least control another device to execute such.

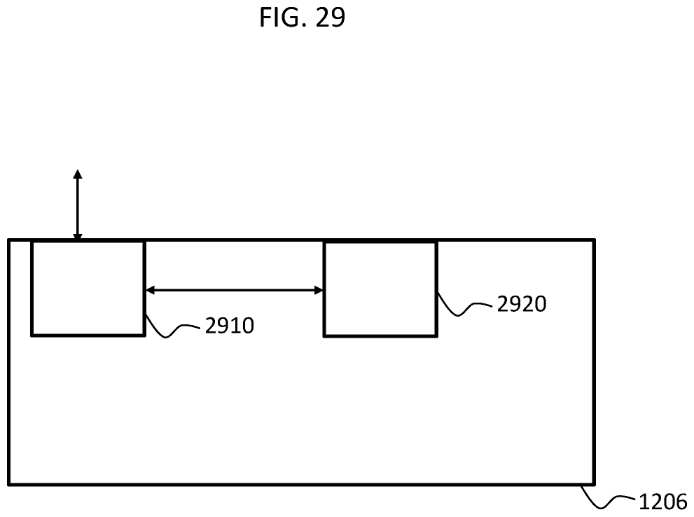

FIG. 29 depicts a functional block diagram that represents an exemplary embodiment of system 1206 that will be utilized to describe the structure of the system 1206. System 1206 includes an input sub-system 2910 configured to receive first data based on a signal response to stimulation applied to a person. (In an exemplary embodiment, the signal response to stimulation applied to the person is in accordance with the teachings detailed herein and/or variations thereof). In an exemplary embodiment, the input subsystem can be a wireless and/or a wired receiver device (e.g., USB port system, wi-fi, RF system, keyboard and software and hardware for such, voice recognition system and hardware and software for such, etc.) that can receive input indicative of the measurements obtained from the implant. It is briefly noted that while the embodiment depicted in FIG. 28 shows the system 1206 in signal communication with the implant, in an alternate embodiment, this may not necessarily be the case. Indeed, this is inferred by the just noted example where a keyboard is utilized. In this regard, the system can be a device that is configured to receive input based on the measurements obtained utilizing the implant where there is a firewall array disconnect between the implant and the system 1206. In an exemplary embodiment, subsystem 2910 can correspond to input interface 1224. The embodiment depicted in FIG. 29 depicts two-way communication capability of the input subsystem 2910. That said, in an exemplary embodiment, there can be only one way to communication.

The system 1206 includes a processor, represented by block 2920 in FIG. 29, which is a processor configured to develop a model based at least in part on the received first data and to extrapolate a biological signal based on a comparison of the model and the received first data.

In an exemplary embodiment, device 2920 is a microprocessor or otherwise a system that includes circuitry or microcircuitry, such as transducers, that can be configured or programmed or can access programming from a memory of the system, to execute the teachings herein. In an exemplary embodiment.

In an exemplary embodiment, the aforementioned processor is a general-purpose processor that is configured to execute one or more the functionalities herein. In some embodiments, the processor includes a chip that is based on machine learning/from machine learning. Any device, system, and/or method that can enable the teachings detailed herein can be utilized in at least some exemplary embodiments.

In an exemplary embodiment, system 1206 can be a personal computer that is programmed to implement at least some of the method actions detailed herein.

In an exemplary embodiment, the processor can instead be a chip assembly configured with circuitry configured to implement one or more of the teachings herein.

In an exemplary embodiment, the processor under chip assembly of the system is configured to receive measurements results in the time domain and/or the frequency domain and utilizing those results, develop a model in accordance with the teachings detailed herein.

In an exemplary embodiment, the system is further configured to utilize the model and compare the model to the measurement data to identify the electrical response resulting from the stimulation that was applied to the recipient (whether such was executed under the control of the system or separately).

As will be understood from the above with respect to the teachings directed to ECAP analysis, in an exemplary embodiment, the system is an ECAP measurement analysis system. Also as will be understood from the above, in an exemplary embodiment, the system is configured to develop the model such that the model closely tracks the first data but cannot and/or does not duplicate the first data. Indeed, in this regard, at least some exemplary embodiments are configured so that the model purposely does not duplicate the first data. In at least some exemplary embodiments, this can be utilitarian with respect to the fact that the goal is to identify the neural response from the overall measurement, where the measurement includes the artefact that results from the initial stimulation that was utilized to cause the neural response, and thus the system is removing the artefact in at least some exemplary embodiments.

Corollary to the above, in an exemplary embodiment, the system is configured to develop the model so that it tracks the first data to a statistically insignificant and/or an effectively insignificant improvable difference relative to other models that the system has or can develop with further development. In this regard, by and effectively insignificant improvable difference, it is to be understood that further improvement would not provide any better effective results with respect to efficacy of the underlying method that is executed utilizing the system.

Consistent with the teachings detailed above, in an exemplary embodiment, the system is an artefact removal system and/or an artefact identification system.

In an exemplary embodiment, the system is configured to and/or the methods detailed herein provide at least X % more accuracy with respect to identifying the underlying neural response from input into the system which is based on and/or is the raw signal measurement from the implant than a system that uses/develops a model based on a double exponential, at least 3 out of 4 times, at least 7 our 8 or 9 or 10 times out of 10 times and/or at least 13, 14, 15, 16, 17, 18, 19, or 20 times out of 20 times. (In some embodiments, the methods and system explicitly exclude a model based on a double exponential.) In an exemplary embodiment, X is 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 800, 900, 1000, 1250, 1500, 1750, 2000, 2250, 2500, 2750, 3000, 3500, 4000, 4500, 5000, 6000, 7000, 8000, 9000, or 10000, or more or any value or range of values therebetween in 1% increments.

In an exemplary embodiment, the accuracy is measured by taking the value of the response obtained using the system/method according to the teachings herein and taking the difference between that value and the value from the contrasting system/method and then dividing that value by the value obtained using the system/method and converting such to a percentage.

In an exemplary embodiment, the system is configured to and/or the methods are such that they provide at least X % more accuracy with respect to identifying the underlying neural response from input into the system which is based on and/or is the raw signal measurement from the implant than a system that is based upon the following at least 3 out of 4 times, at least 7 our 8 or 9 or 10 times out of 10 times and/or at least 13, 14, 15, 16, 17, 18, 19, or 20 times out of 20 times: (i) Alternating Stimulation polarity (ii) a regime that relies on the premise that the biological potential being recorded is independent of the polarity of the electrical stimulation, (iii) a regime that utilizes two subsequent stimulations (of opposing polarity) that are summed and the stimulation artefact cancels but the biological potential does not, (iv) forward masking, (v) a regime that relies on the behavior of some bio-potentials known as a refractory period, (vi) a regime that records after a masker-probe pair, (vii) a regime that provides a measurement which includes the stimulation artefact, but without a neural response in response to the probe, (viii) artefact scaling, (ix) a regime that relies on forward masking technique of subtracting a masker only stimulus measurement from a masker-probe stimulus measurement to obtain the probe only stimulation artefact, (x) a regime that utilizes tri phasic stimulation and/or (xi) a regime that seeks to suppress the stimulation artefact, by adding a third phase of stimulation of opposite polarity to the second phase of stimulation, rather than eliminate it via the measurement paradigm.

In an exemplary embodiment, the systems configured and/or the methods detailed herein provides at least X % of a value difference respect to identifying the underlying neural response from input into the system which is based on and/or is the raw signal measurement from the implant than a system that uses/develops the competing models detailed above, at least 3 out of 4 times, at least 7 our 8 or 9 or 10 times out of 10 times and/or at least 13, 14, 15, 16, 17, 18, 19, or 20 times out of 20 times. (In some embodiments, the methods and system explicitly exclude a model based on a double exponential.)

In an exemplary embodiment, difference is measured by taking the value of the response obtained using the system/method according to the teachings herein and taking the difference between that value and the value from the contrasting system/method and then dividing that value by the value obtained using the competing difference and converting such to a percentage.

In an exemplary embodiment, the system is configured such that and/or the methods detailed herein provide, over a time period spanning 0.1, 0.15, 0.2, 0.3, 0.4, 0.5, 0.6, 0.7. 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 milliseconds or any value or range of values therebetween in 0.01 milliseconds, starting a time T after the stimulus begins and/or ends and/or a medium time, where T is 0.01, 0.02, 0.03, 0.04, 0.05, 0.06, 0.07, 0.08, 0.09, 0.1, 0.11, 0.12, 0.13, 0.14, 0.15, 0.175, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40 or more milliseconds, or any value or range of values therebetween in 0.01 milliseconds, average deviation (mean, median and/or mode) from the data recorded from the measurements of the artefact model is no more than Z percent, where Z is 0.01, 0.02, 0.03, 0.04, 0.05, 0.06, 0.07, 0.08, 0.09, 0.1, 0.11, 0.12, 0.13, 0.14, 0.15, 0.175, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.45, 0.50, 0.55, 0.60, 0.65, 0.7, 0.75, 0.8, 0.9, 1, 1.25, 1.5, 1.75, 2, 2.5, 3, 3.5, 4, 4.5, 5, 5.5, 6, 7, 8, 9, 10, 11, 12, 13, 14, 15, 19, 17, 18, 19, or 20, or any value or range of values therebetween in 0.01% increments, where the percentage is measured from the difference of the model to the recorded data divided by the recorded data then converted to a percentage, at least 3 out of 4 times, at least 7 our 8 or 9 or 10 times out of 10 times and/or at least 13, 14, 15, 16, 17, 18, 19 or 20 times out of 20 times.

In an exemplary embodiment, the methods and systems herein do not utilize linearization techniques to develop the model. In this regard, some embodiments explicitly avoid all sequential linear fits. In some embodiments, the teachings detailed herein explicitly avoid utilizing one, two, three, four, five or more sequential linear fits to develop the model and/or the equivalence thereof. In at least some exemplary embodiments, the teachings detailed herein explicitly avoid the utilization of a slew rate, such as that which is limited by the amplifier and/or amplifier system of the implanted component. In at least some exemplary embodiments, any residuals that results from the difference between the model and the recorded data is not merely a smaller exponential.

Again, at least some embodiments involve fitting a true constant phase model, as opposed to successfully fitting more and more exponential decays. In this regard, at least some embodiments avoid the actions of fitting one, two, three, four, five, six, seven, eight, nine, 10, 11, 12, 13, 14, 15, 16, 17, 18, 19, or 20 or more exponential decays.

In an exemplary embodiment, some embodiments utilize PCA. In at least some exemplary embodiments, the teachings detailed herein involve a digital technique for estimating and subtracting the stimulation artefact utilizing a mathematical model and numerical methods, with no additional hardware beyond that which is utilized to obtain the measurement data in the first instance and, in embodiments utilizing computers or other processors or chips, etc., the device is to implement those mathematical models and numerical methods to develop the model. Accordingly, in an exemplary embodiment, the system 1206 and the communication regime between the hardware of the system and the implant and the implant are the only components that are utilized to execute at least some methods detailed herein.

It is noted that in at least some exemplary embodiments, there are methods that include evaluating the neural response that is identified utilizing at least some of the teachings detailed herein, and then fitting or otherwise adjusting the prostheses to the recipient based on the evaluation of the neural response. In an exemplary embodiment, the neural response data is utilized in conjunction with threshold and/or comfort levels to develop a map for a cochlear implant. The map is then loaded into the memory of the cochlear implant, and then the cochlear implant evokes hearing percepts based on captured sound based on the map. Accordingly, at least some embodiments include cochlear implants that include map data or otherwise are programmed based at least in part one data that is based on the utilizations of the teachings detailed herein.

Some embodiments include evaluating the neural response data that is obtained according to the teachings detailed herein or variations thereof, and, based on the evaluation, repositioning the electrode array or the electrodes that are utilized to obtain the read data. In an exemplary embodiment, this can correspond to adjusting a cochlear implant electrode array that has been inserted in a cochlea. In an exemplary embodiment, there are methods that include, during surgery, inserting the electrode array into the cochlea, activating the electrode array in accordance with the teachings detailed herein, evaluating the neural response data, repositioning the electrode array, again activating the electrode array, evaluating the new neural response data, and someone, until a desired neural response is achieved, and determining, based on that neural response, that the electrode array is in a position that has utilitarian value or otherwise will not benefit in a meaningful manner from further adjustments with respect to the location thereof. At that point, some exemplary embodiments, or shortly thereafter, the surgery will be commenced and the incision into head is closed and the cochlear implant electrode array is intended to remain at the location of its last position.

That said, as noted above, some embodiments have nothing to do with implantation. Accordingly, at least some exemplary embodiments are directed towards evaluating the neural response after the implant has stabilized, etc. This can correspond to, for example, after the development of any scar tissue that would be present resulting from the implantation.

Any method action detailed herein corresponds to a disclosure of a device and/or a system for executing that method action. Any disclosure of any method of making an apparatus detailed herein corresponds to a resulting apparatus made by that method. Any functionality of any apparatus detailed herein corresponds to a method having a method action associated with that functionality. Any disclosure of any apparatus and/or system detailed herein corresponds to a method of utilizing that apparatus and/or system. Any feature of any embodiment detailed herein can be combined with any other feature of any other embodiment detailed herein providing that the art enables such, unless such is otherwise noted.

Any disclosure herein of a method of making a device herein corresponds to a disclosure of the resulting device. Any disclosure herein of a device corresponds to a disclosure of making such a device.

Any one or more elements or features disclosed herein can be specifically excluded from use with one or more or all of the other features disclosed herein.

While various embodiments of the present invention have been described above, it should be understood that they have been presented by way of example only, and not limitation. It will be apparent to persons skilled in the relevant art that various changes in form and detail can be made therein without departing from the scope of the invention.

1. A method, comprising:
applying electrical stimulation to a recipient;
obtaining from read electrodes read data resulting from the applied stimulation;
obtaining an artefact model based at least in part on the read data; and
obtaining neural response data by comparing the read data to the artefact model.

2. (canceled)

3. The method of claim 1, wherein:
the application of electrical stimulation and the obtaining of the read data occurs at a cochlea of a person.

4. (canceled)

5. The method of claim 1, wherein the artefact model is based on a constant phase model.

6. The method of claim 1, wherein the artefact model is based on a true constant phase model.

7. The method of claim 1, wherein the artefact model does not rely on the results of a double exponential.

8. The method of claim 1, wherein the action of obtaining neural response data is executed by subtracting the artefact model from the read data.

9. The method of claim 1, further comprising accounting for, at least in part, noise that influenced the results of the obtained artefact model.

10-14. (canceled)

15. A method, comprising:
developing a recipient-specific electrical stimulation artefact model.

16. (canceled)

17. The method of claim 15, wherein:
the model is based on a constant phase model.

18. The method of claim 15, wherein the action of developing the model includes:
obtaining one or more temporally and/or frequency based dataset(s) from sensor(s) attached to the recipient;
developing various iterations of embryonic models based at least in part on the obtained one or more dataset(s);
comparing at least some of the respective various iterations of the embryonic models to the one or more dataset(s); and
identifying at least one respective embryonic model that tracks the one or more dataset(s) in a predetermined manner,
wherein the model is based at least in part on the identified at least one respective embryonic model.

19. (canceled)

20. The method of claim 15, wherein the action of developing the model includes:
obtaining one or more temporally and/or frequency based dataset(s) from sensors attached to the recipient;
developing various iterations of embryonic models, all of which are intended to be different from the dataset(s) and
using one of the iterations as a basis for the model.

21. (canceled)

22. The method of claim 21, wherein:
the comparison yields a difference between the temporally and/or frequency based dataset(s) and the model, the difference being the neural response.

23. The method of claim 15, wherein:
the action of developing the model includes obtaining one or more temporally and/or frequency based dataset(s) from sensors attached to the recipient and developing the model at least in part based thereon; and
the method further comprises comparing the developed model to the dataset(s) to identify a neural response.

24. (canceled)

25. The method of claim 19, wherein:
the temporally and/or frequency based dataset(s) is/are dataset(s) where the neural repose is overwhelmed by the artefact.

26. (canceled)

27. An electrical response stimulation measurement system, comprising:
an input sub-system configured to receive first data based on a signal response to stimulation applied to a person; and
a processor and/or chip assembly configured to develop a model based at least in part on the received first data and to extrapolate a biological signal based on a comparison of the model and the received first data.

28. The system of claim 27, wherein:
the system is an ECAP measurement analysis system.

29. The system of claim 27, wherein:
the system is configured to develop the model that closely tracks the first data but cannot and/or does not duplicate the first data.

30. The system of claim 27, wherein:
the system is configured to develop the model so that it tracks the first data to a statistically insignificant and/or an effectively insignificant improvable difference relative to other models that the system has or can develop with further development.

31. (canceled)

32. The system of claim 27, wherein:
the system is a stimulation artefact removal system and/or a stimulation artefact identification system.

33. The system of claim 27, wherein:
the system provides at least 30% more accuracy than any of the prior art systems at least 3 out of 4 times.

34-35. (canceled)
